(19) United States
(12) Patent Application Publication
(10) Pub. No.: US 2025/0281297 A1
(43) Pub. Date: Sep. 11, 2025

(54) VENTRICLE TETHERING

(71) Applicant: EDWARDS LIFESCIENCES CORPORATION, Irvine, CA (US)

(72) Inventors: Stephen Cournane, Severn, MD (US); Felino V. Cortez, JR., Bowie, MD (US); Sai Prasad Uppalapati, Plano, TX (US); Stephen Epstein, Millersville, MD (US); Ashley Nicolette Hinga, Eldersburg, MD (US); Luke Anthony Zanetti, Parkton, MD (US); Nancy Ling Chung, Edgewater, MD (US)

(21) Appl. No.: 19/220,506

(22) Filed: May 28, 2025

Related U.S. Application Data
(63) Continuation of application No. PCT/US2023/079598, filed on Nov. 14, 2023.
(60) Provisional application No. 63/385,191, filed on Nov. 28, 2022.

(51) Int. Cl. A61F 2/24

(52) U.S. Cl. CPC A61F 2/2487; A61F 2220/0008

(57) ABSTRACT

A method comprises coupling a first anchor to an annulus of a heart valve that is between a heart atrium and a heart ventricle, and extending a portion of a tether within the heart ventricle from the first anchor to a portion of a ventricular heart wall of the heart ventricle between an apex and a basal portion of the ventricular heart wall. The method can include positioning a second anchor over an externally oriented surface of the portion of the ventricular heart wall, and coupling the tether to the second anchor while the second anchor is over the externally oriented surface of the ventricular heart wall.

### CROSS REFERENCE TO RELATED APPLICATIONS

This application is a continuation of International Patent Application No. PCT/US2023/079598, filed Nov. 14, 2023, which claims the benefit of U.S. Patent Application No. 63/385,191, filed Nov. 28, 2022, the entire disclosures all of which are incorporated by reference for all purposes.

### BACKGROUND

The present disclosure generally relates to the field of cardiac repairs, and more particularly to minimally invasive cardiac repair operations. Cardiac abnormalities can contribute to various heart conditions, adversely affecting supply of oxygenated blood to the rest of the body. Insufficient supply of oxygen can produce any number of symptoms, adversely affecting quality of life. Cardiac repair procedures can be performed to treat the abnormalities and alleviate the symptoms.

### SUMMARY

Described herein are methods, devices and/or systems related to reducing or reversing heart ventricular remodeling. A tether system can be configured to couple a first portion of a heart to a second portion of the heart to facilitate reducing or reversing remodeling of a heart ventricle. In some instances, a tether system can be configured to couple a portion of a heart valve annulus to a portion of a ventricular heart wall. For example, a tether system can comprise a first anchor configured to be deployed to an atrioventricular valve annulus, a second anchor configured to be deployed to an externally oriented surface of the ventricular wall, and a tether comprising a portion configured to extend within the heart ventricle to couple to first and second anchors to one another. In some instances, a tether system can be configured to couple a portion of a septal wall to a portion of a ventricular heart wall. For example, a tether system can comprise a first anchor configured to be deployed to the septal wall, a second anchor configured to be deployed to an externally oriented surface of the ventricular heart wall, and a tether comprising a portion configured to extend within the heart ventricle to couple to first and second anchors to one another. In some instances, a first portion of a ventricular heart wall can be coupled to a second portion of a ventricular heart wall. For example, a tether system can comprise a first anchor configured to be deployed to an externally oriented surface of the first portion of the ventricular heart wall, a second anchor configured to be deployed to an externally oriented surface of the second portion of the ventricular heart wall, and a tether comprising a portion configured to extend within the heart ventricle to couple to first and second anchors to one another. In some instances, a tether system can be deployed to couple a papillary muscle to a ventricular heart wall, including an apical portion of the ventricular heart wall. For example, a tether system can comprise a first anchor configured to be deployed to the papillary muscle, a second anchor configured to be deployed to an externally oriented surface of the ventricular heart wall, and a tether comprising a portion configured to extend within the heart ventricle to couple the first and second anchors to one another.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 provides a cutaway view of a heart, and a side view of a tether system deployed into the heart and configured to couple a portion of a heart valve annulus and a portion of a ventricular heart wall to one another, in accordance with one or more examples.

FIG. 2 provides a cutaway view of a heart, and a side view of another tether system deployed into the heart and configured to couple a portion of a heart valve annulus and a portion of a ventricular heart wall to one another, in accordance with one or more examples.

FIG. 3 provides a flow diagram of a process for deploying a tether system configured to couple a heart valve annulus and a ventricular heart wall to one another, in accordance with one or more examples.

FIG. 4 provides a cutaway view of a heart, and a side view of a tether system deployed into the heart and configured to couple a portion of a septal wall of a heart ventricle and a portion of a ventricular heart wall of the heart ventricle to one another, where the tether system comprises a tether configured to extend directly between the septal wall and the ventricular heart wall, in accordance with one or more examples.

FIG. 5 provides a cutaway view of a heart, and a side view of a tether system deployed into the heart and configured to couple a portion of a septal wall of a heart ventricle and a portion of a ventricular heart wall of the heart ventricle to one another, where the tether system comprises a tether configured to form a loop within the heart ventricle between the septal wall and the ventricular heart wall, in accordance with one or more examples.

FIG. 6 provides a cutaway view of a heart, and a side view of a tether system deployed into the heart and configured to couple a portion of a septal wall of a heart ventricle and a portion of a ventricular heart wall of the heart ventricle to one another, where the tether system comprises a tether configured to form a loop within the heart ventricle between the septal wall and the ventricular heart wall, in accordance with one or more examples.

FIG. 7 provides a flow diagram of a process for deploying a tether system configured to couple a portion of a septal wall of a heart ventricle and a portion of a ventricular heart wall of the heart ventricle to one another, in accordance with one or more examples.

FIG. 8 provides a cutaway view of a heart and a side view of a tether system deployed into the heart and configured to couple a papillary muscle of a heart ventricle and a ventricular heart wall of the heart ventricle to one another, in accordance with one or more examples.

FIG. 9 provides a flow diagram of a process for deploying a tether system configured to couple a papillary muscle of a heart ventricle and a ventricular heart wall of the heart ventricle to one another, in accordance with one or more examples.

FIG. 10 provides a cutaway view of a heart and a side view of a tether system deployed into the heart and configured to couple a first portion of a ventricular heart wall of a heart ventricle and a second portion of the ventricular heart wall to one another via a papillary muscle of the heart ventricle, in accordance with one or more examples.

FIG. 11 provides a flow diagram of a process for deploying a tether system configured to couple a first portion of a ventricular heart wall of a heart ventricle and a second portion of the ventricular heart wall to one another via a papillary muscle of the heart ventricle, in accordance with one or more examples.

FIG. 12 provides a perspective view of a tether delivery system, in accordance with one or more examples.

FIGS. 13A, 13B, 13C, 13D and 13E provide cutaway views of the heart and various steps of deploying a tether using the tether delivery system described with reference to FIG. 12, in accordance with one or more examples.

### DETAILED DESCRIPTION

The headings provided herein are for convenience only and do not necessarily affect the scope or meaning of the claims.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

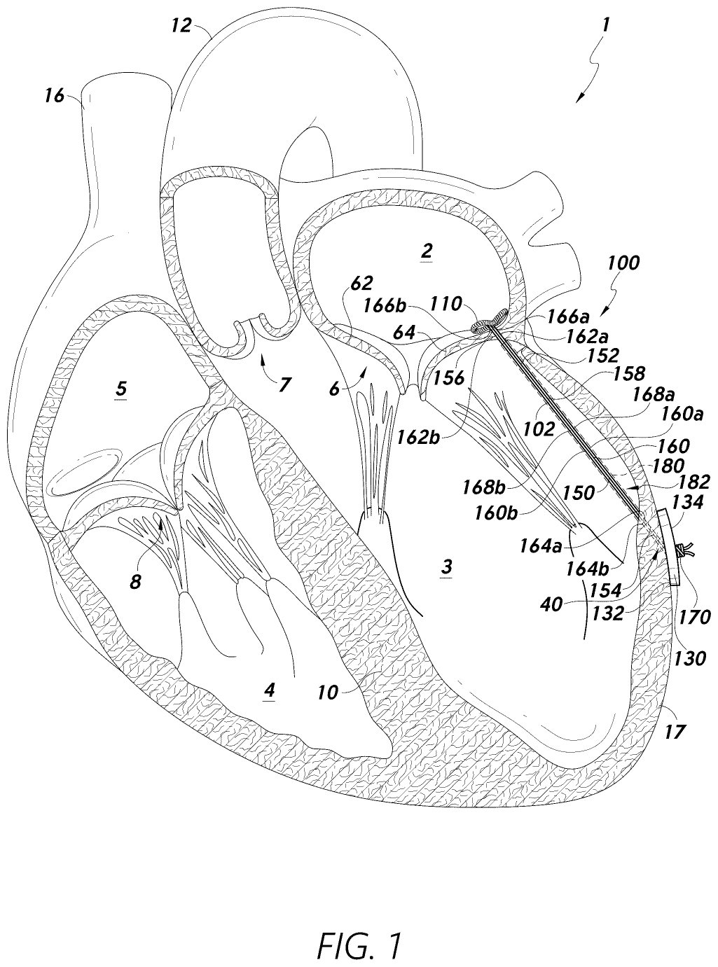

FIG. 1 shows certain anatomical features of human vasculature, including various features of a human heart 1. The heart 1 includes four chambers, namely the left atrium 2, the left ventricle 3, the right ventricle 4, and the right atrium 5. A wall of muscle, referred to as the septal wall 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve separates the right ventricle 4 and the pulmonary trunk or artery, controlling blood flow therebetween.

In a healthy heart, the heart valves can properly open and close in response to a pressure gradient present during various stages of the cardiac cycle (e.g., relaxation and contraction) to at least partially control the flow of blood to a respective region of the heart and/or to blood vessels. Deoxygenated blood arriving from the rest of the body generally flows into the right side of the heart for transport to the lungs, and oxygenated blood from the lungs generally flows into the left side of the heart for transport to the rest of the body. During ventricular diastole, deoxygenated blood arrive in the right atrium 5 from the inferior vena cava and superior vena cava 16 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary trunk for transport to the lungs (e.g., via the left and right pulmonary arteries), and oxygenated blood can flow from the left ventricle 3 to the aorta 12 for transport to the rest of the body.

The shape of a heart ventricle may change over time due to any number of conditions. Remodeling of the ventricular wall can progress over time due to volume overload, pressure overload, and/or myocardial injury. For example, mitral valve dysfunction can contribute to volume overload that is exerted on the left ventricular wall. Elevated stress on the left ventricular heart wall, such as during diastole, can cause the shape of the left ventricular wall to change. Remodeling of the ventricular wall can contribute to displacement of the papillary muscles and tenting of the atrioventricular valve. For example, remodeling of the left ventricular wall can cause displacement of one or more papillary muscles of the left ventricle and tenting of the mitral valve, such as during systole.

The present disclosure provides systems, devices, and methods relating to tether systems configured to be deployed into the heart to facilitate reversal of and/or reduction in the shape remodeling of a heart ventricle. Although the systems, devices, and methods described herein are referred primarily to as being used to deploy one or more tether systems into a left heart ventricle, it will be understood that the systems, devices, and methods can be applied to deploy one or more tether systems to a right ventricle.

In some instances, a tether system can be deployed into the heart to reverse and/or reduce remodeling a heart ventricular wall, such as a left ventricular wall. Reversal and/or reduction in remodeling of the heart ventricular wall can facilitate return of one or more papillary muscles to their respective original location. Reversal and/or reduction in remodeling of the left heart ventricular wall can facilitate return of one or more papillary muscles of the left ventricle to their original location. In some instances, relocating the papillary muscle can facilitate improvement in valve leaflet coaptation, for example reducing mitral valve regurgitation. In some instances, one or more tether systems described herein can be deployed to reduce or eliminate long term mitral valve regurgitation, such as in patients who have undergone multi-vessel coronary artery bypass graft (CABG) procedures and/or patients who experience moderate to severe ischemic functional mitral valve regurgitation.

In some instances, coupling a heart valve annulus to a portion of a ventricular heart wall can facilitate pulling the portion of the ventricular heart wall upward and/or inward. For example, a tether system can comprise a first anchor configured to be deployed to an atrioventricular valve annulus, a second anchor configured to be deployed to an externally oriented surface of the ventricular wall, and a tether comprising a portion configured to extend within the heart ventricle between the first and second anchors to couple the first and second anchors to one another.

In some instances, a portion of a septal wall can be coupled to a portion of a ventricular heart wall to facilitate pulling the portion of the ventricular heart wall inward and/or upward. For example, a tether system can comprise a first anchor configured to be deployed to the portion of the septal wall, a second anchor configured to be deployed to an externally oriented surface of the portion of the ventricular heart wall, and a tether comprising a portion configured to extend within the heart ventricle between the first and second anchors to couple the first and second anchors to one another.

In some instances, a first portion of a ventricular heart wall can be coupled to a second portion of the ventricular heart wall to facilitate pulling the second portion of the ventricular heart wall inward. The second portion of the ventricular heart wall can be at a lower position relative to that of the first portion, for example an inferior position relative to that of the first portion. A tether system can comprise a first anchor configured to be deployed to a first portion of a ventricular heart wall, a second anchor configured to be deployed to an externally oriented surface of a second portion of the ventricular heart wall, and a tether comprising a portion configured to extend within the heart ventricle between the first and second anchors to couple the first and second anchors to one another. The portion of the tether configured to be within the heart ventricle can form a partial loop around a papillary muscle of the heart ventricle to facilitate pulling the second portion of the ventricular heart wall inward. In some instances, the first anchor can be over an externally oriented surface of the first portion of the ventricular heart wall.

In some instances, one or more tether systems can be deployed to couple a papillary muscle to a ventricular heart wall. In some instances, a tether system can comprise a first anchor configured to be deployed to a papillary muscle, a second anchor configured to be deployed to an externally oriented surface of a portion of the ventricular heart wall, and a tether comprising a portion configured to extend within the heart ventricle between the first and second anchors to couple the first and second anchors to one another. In some instances, the first anchor can be coupled to an apical portion of the heart ventricular wall. The tether system can be configured to pull relocate the papillary muscle, such as pulling the papillary muscle and portion of the ventricular wall adjacent to the papillary muscle inward.

A procedure as described herein to improve or restore the shape of a ventricular heart wall can comprise deploying one or more tether systems as described herein. The one or more tether systems can be delivered while the heart is beating. In some instances, one or more tether systems can be delivered using a minimally invasive technique. For example, the procedure to deliver one or more tether systems can be minimally invasive beating heart procedure. Such procedures can be used to reduce and/or eliminate heart ventricular wall remodeling. Reducing and/or eliminating heart ventricular wall remodeling can improve mitral regurgitation, such as functional mitral regurgitation. The tether systems described herein can be delivered using procedures that can be performed off-pump, allowing for less invasive procedures and/or facilitating appropriate adjustments in lengths of the tether systems, such as compared to on-pump procedures.

As used herein, a basal portion of a ventricular heart wall can refer to a top third of the ventricular heart wall, such as a superior third of the ventricular heart wall. A mid-portion of a ventricular heart wall can refer to a middle third of the ventricular heart wall. An apical portion of a ventricular heart wall can refer to a lower third of the ventricular heart wall, such as an inferior third of the ventricular heart wall. A basal portion of a septal wall can refer to a top third of the septal wall, such as a superior third of the septal wall. A mid-portion of a septal wall can refer to a middle third of the septal wall. An apical portion of a septal wall can refer to a lower third of the septal wall, such as an inferior third of the septal wall.

Although tether systems are described herein primarily as comprising a suture and/or cord and a bulky-knot type anchor, any number of different mechanical tethering components can be applied. In some instances, one or more portions of the tether system, such as a tether and/or anchor portion, can comprise a material that is rigid, semi-rigid or flexible. In some instances, a tether of the tether system can comprise a length at least about twice as long as the tether is wide. For example, a tether tail of the tether can comprise a length at least about twice as long as the tether tail is wide. In some instances, an anchor, including a first and/or second anchor as described herein, can comprise any number of mechanical anchors, such as a T-fastener and/or hook. In some instances, a tether system can comprise a solid rod, including a rod comprising at least a portion that is rigid, semirigid, or flexible, and respective mechanical anchor coupled to each of the two distal end portions of the solid rod. For example, a tether system can comprise a T-fastener coupled to each of two distal end portions of a solid rod. It will be understood that the anchors described herein can be deployed in any order.

The methods, operations, steps, etc. described herein can be performed on a living animal or on a non-living cadaver, cadaver heart, anthropomorphic ghost, simulator (e.g., with the body parts, tissue, etc. being simulated), etc. For example, methods for treating a patient include methods for simulating the treatment on a simulated patient or anthropogenic ghost, which can include any combination of physical and virtual elements. Examples of physical elements include human or animal cadavers; any portions thereof, including organ systems, whole organs, or tissue; and manufactured elements, which can simulate the appearance, texture, resistance, or other characteristic. Virtual elements can include visual elements provided on a screen, or projected on a surface or volume, including virtual reality and augmented reality implementations. Virtual elements can also simulate other sensory stimuli, including sound, feel, and/or odor.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

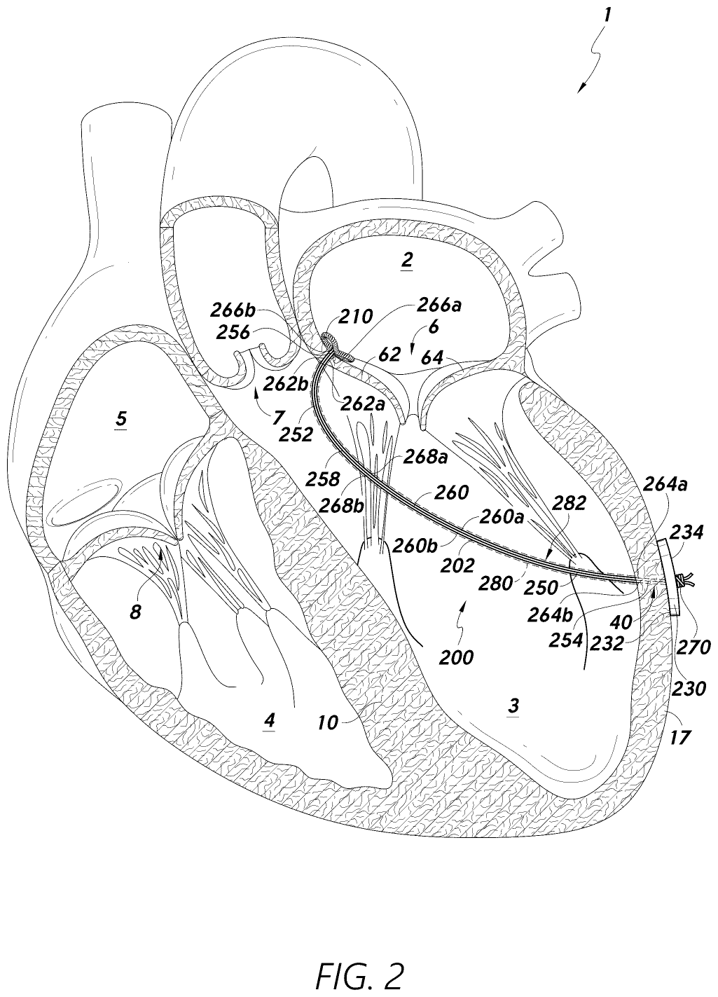

FIGS. 1 and 2 show tether systems 100, 200 deployed in the heart 1 for coupling a portion of a heart valve annulus and a portion of a ventricular heart wall 17 to one another. Each of the tether systems 100, 200 can comprise a first anchor 110, 210, a second anchor 130, 230, and a tether 150, 250 configured to couple the respective first anchor 110, 210 and the second anchor 130, 230 to one another. The first anchor 110, 210 can be configured to be coupled to an annulus of a heart valve. In some instances, the heart valve can be between a heart atrium and a heart ventricle. For example, the first anchor 110, 210 can be coupled to a heart valve between the left atrium 2 and the left ventricle 3, such as the mitral valve 6. The second anchor 130, 230 can be configured to be positioned over an externally oriented surface of a portion of a ventricular heart wall 17 of the heart ventricle, such as a ventricular heart wall 17 of the left ventricle 3. The second anchor 130, 230 can be positioned over the externally oriented surface between an apex and a basal portion of the ventricular heart wall 17. In some instances, the tether systems 100, 200 can be configured to couple an annulus of a heart valve controlling blood flow into a heart ventricle to a portion of a ventricular heart wall of the heart ventricle so as to pull the portion of the ventricular heart wall upward toward the annulus. Relocating the portion of the ventricular heart wall upward and/or inward toward the annulus can reduce or reverse remodeling of the portion of the ventricular heart wall. For example, the tether systems 100, 200 can be configured to couple an annulus of the mitral valve 6 to a portion of the ventricular heart wall 17 of the left ventricle 3.

The position on the annulus to which a first anchor is deployed can be selected to provide the desired ventricular wall remodeling. In some instances, for example as shown in FIG. 1, the first anchor 110 can be deployed to a portion of the annulus adjacent to a posterior leaflet 64 of the mitral valve 6. Coupling a portion of the ventricular heart wall to a portion of the annulus adjacent to a posterior leaflet 64 of the mitral valve 6 can facilitate pulling the portion of the ventricular heart wall upward toward the portion of the annulus. In some instances, for example as shown in FIG. 2, the first anchor 210 can be deployed to a portion of the annulus adjacent to an anterior leaflet 62 of the mitral valve 6. Coupling a portion of the ventricular heart wall to a portion of the annulus adjacent to an anterior leaflet 62 of the mitral valve 6 can facilitate pulling the portion of the ventricular heart wall upward and inward toward the portion of the annulus.

In some instances, the portion of the ventricular heart wall over which the second anchor 130, 230 is placed can be in a mid-portion of the ventricular heart wall. In some instances, the portion of the ventricular heart wall over which the second anchor 130, 230 is placed can be between an apical portion and a mid-portion of the ventricular heart wall. In some instances, the portion of the ventricular heart wall over which the second anchor 130, 230 is placed can be adjacent to a portion of the ventricular heart wall from which a papillary muscle extends. In some instances, the portion of the ventricular heart wall over which the second anchor 130, 230 is placed can be adjacent to a portion of the ventricular heart wall from which an anterolateral papillary muscle extends.

The first anchor 110, 210 can be configured to be disposed over an atrial facing portion of the portion of the heart valve annulus, such as the annulus of the mitral valve 6. As described herein, the second anchor 130, 230 can be configured to be positioned over an externally oriented surface of a ventricular heart wall, such as the ventricular heart wall 17 of the left ventricle 3. In some instances, each of the tethers 150, 250 can comprise a first distal portion 152, 252 configured to extend through the heart valve annulus, such as from a ventricular facing surface to the atrial facing surface of the annulus. The first distal portion 152, 252, such as a first distal end 156, 256, of the tether 150, 250 can be coupled to the first anchor 110, 210. The tether 150, 250 can comprise another portion configured to be disposed through the ventricle, such as the left ventricle 3. For example, a medial portion 158, 258 of the tether 150, 250 can be configured to be disposed within the left ventricle 3. A second distal portion 154, 254 can be configured to extend through the ventricular heart wall 17, such as from a ventricular facing surface to the externally oriented surface such that the tether 150, 250 can be coupled to the second anchor 130, 230. For example, the second distal portion 154, 254 can extend through an incision and/or opening 40 made in the ventricular heart wall 17.

In some instances, the second anchor 130, 230 can comprise a first surface 132, 232 configured to be oriented toward the ventricular heart wall and a second surface 134, 234 configured to be oriented away from the ventricular heart wall. In some instances, the second anchor 130, 230 can comprise a pad configuration. In some instances, the second anchor 130, 230 can comprise a support platform configured to facilitate maintaining the second distal portion 154, 254 of the tether 150, 250 at the desired position on the ventricular heart wall. The first surface 132, 232 can be configured to be over and in contact with the ventricular heart wall. In some instances, the second anchor 130, 230 can comprise a pad configuration. In some instances, the second anchor 130, 230 can comprise a support platform configured to facilitate distribution of the tension exerted by the tether 150, 250 upon the ventricular heart wall to improve the reduction in remodeling achieved. For example, the support platform can facilitate distribution of the tension exerted by the tether 150, 250 upon the ventricular heart wall over a larger area to allow a larger area of the ventricular heart wall to be reverse remodeled. The second anchor 130, 230 can be a support platform having a first surface 132, 232 configured to be over and in contact with the ventricular heart wall 17 and a second opposing surface 134, 234 configured to be oriented away from the from the ventricular heart wall 17.

In some instances, the first anchor 110, 210 and the tether 150, 250 can be an integral piece. In some instances, the first anchor 110, 210 and the tether 150, 250 can be formed using the same material. The first anchor 110, 210 and the tether 150, 250 can each comprise a respective portion of a surgical cord and/or suture. For example, a portion of a suture 102, 202 can form the first anchor 110, 210. Another portion of the suture 102, 202 can form the tether 150, 250. As described in further detail herein, in some instances, the first anchor 110, 210 can comprise a bulky-knot. For example, a portion of the suture 102, 202 can be deployed through the portion of the heart valve annulus to form the first anchor 110, 210 over and in contact with an atrium-facing surface of the annulus. Another portion of the suture 102, 202 can form the tether 150, 250, for example extending through the annulus, the ventricle, and a portion of the ventricular wall, such that the suture 102, 202 can be coupled to the second anchor 130, 230 positioned over the externally oriented surface of the ventricular wall. For example, the first anchor 110, 210 can be formed over a surface of the mitral valve annulus oriented toward the left atrium. The tether 150, 250 can extend through the mitral valve annulus, left ventricle and a portion of the left heart ventricular wall adjacent to the anterolateral papillary muscle to couple to the second anchor 130, 230. The second anchor 130, 230 can be positioned over the portion of the ventricular heart wall adjacent to a portion of the heart ventricular wall from which an anterolateral papillary muscle extends.

In some instances, each of the tethers 150, 250 can comprise respective tether tails 160, 260. For example, the tethers 150, 250 can each comprise a pair of tether tails 160a, 160b, 260a, 260b. For example, the tether tails 160a, 160b, 260a, 26ob can comprise a respective first distal portion 162a, 162b, 262a, 262b configured to extend through the portion of the annulus. In some instances, first distal ends 166a, 166b, 266a, 266b can be coupled to the first anchors 110, 210. A second distal portion 164a, 164b, 264a, 264b of each of the tether tails 160a, 160b, 260a, 260b can be configured to extend through the portion of the ventricular heart wall. In some instances, the second distal portions 164a, 164b, 264a, 264b can be configured to be coupled to the respective second anchors 130, 230. For example, the second distal portions 164a, 164b, 264a, 264b can be configured to extend through the respective second anchors 130, 230 and form the knot 170, 270 for coupling the tethers 150, 250 to the second anchors 130, 230. A medial portion 168a, 168b, 268a, 268b of the tether tails 160a, 160b, 260a, 26ob can be configured to be disposed within the heart ventricle.

In some instances, the tether systems 100, 200 can optionally comprise an elongate tube 180, 280 comprising a lumen 182, 282 configured to receive at least a portion of the tether 150, 250 extending within the heart ventricle. The elongate tube 180, 280 can be configured to prevent or reduce damage to heart tissue due to contact between the tether 150, 250 and the heart tissue.

It will be understood that a tether system as described herein can be applied to either atrioventricular heart valve. For example, a first anchor can be deployed to be coupled to a tricuspid valve. A second anchor can be deployed to an externally oriented surface portion of a right ventricular heart wall. A tether can comprise a portion configured to extend within the right ventricle to couple the first anchor and the second anchor to one another. The tether system for coupling the tricuspid valve annulus to the right ventricular heart wall can comprise one or more features of the tether systems 100, 200 described with reference to FIGS. 1 and 2.

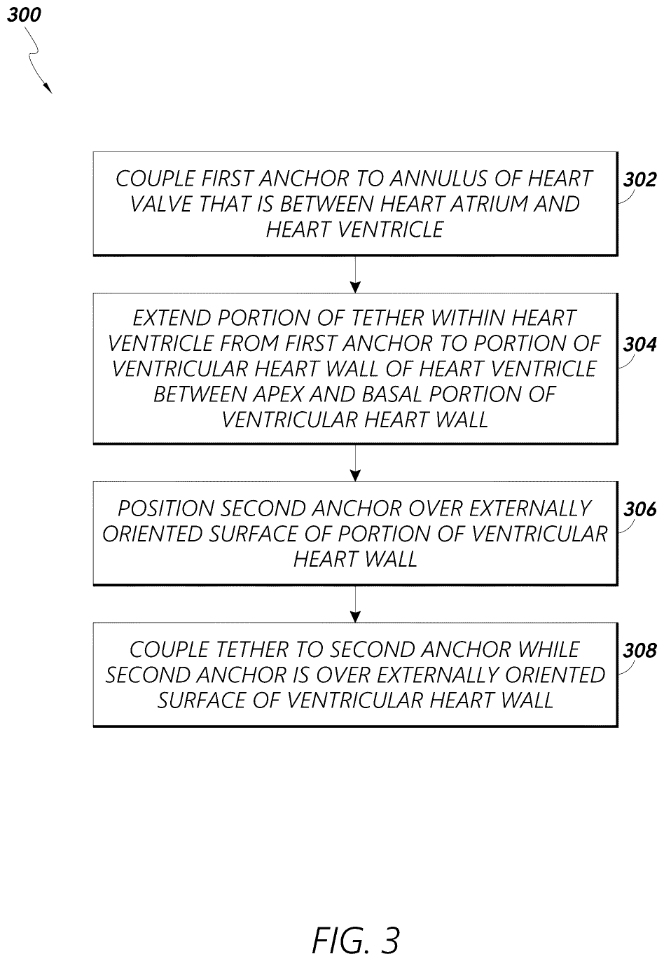

FIG. 3 is a process flow diagram showing an example of a process 300 for deploying a tether system as described herein, including one or more of the tether systems 100, 200 described with reference to FIGS. 1 and 2. In block 302, the process can involve coupling a first anchor to an annulus of a heart valve that is between a heart atrium and a heart ventricle. In block 304, the process can involve extending a portion of a tether within the heart ventricle from the first anchor to a portion of a ventricular heart wall of the heart ventricle between an apex and a basal portion of the ventricular heart wall. In block 306, the process can involve positioning a second anchor over an externally oriented surface of the portion of the ventricular heart wall. In block 308, the process can involve coupling the tether to the second anchor while the second anchor is over the externally oriented surface of the ventricular heart wall.

In some instances, extending the portion of the tether from the first anchor to the portion of the heart ventricular heart can comprise extending the tether from the first anchor to a mid-portion of the ventricular heart wall. In some instances, extending the portion of the tether from the first anchor to the portion of the heart ventricular heart can comprise extending the tether from the first anchor to a portion of the ventricular heart wall between an apical portion and a mid-portion of the ventricular heart wall. In some instances, extending the portion of the tether from the first anchor to the portion of the ventricular heart wall can comprise extending the tether from the first anchor to a portion of the ventricular heart wall adjacent to a papillary muscle.

In some instances, coupling the first anchor to the annulus of the heart valve can comprise coupling the first anchor to a mitral valve annulus. Extending the portion of the tether within the heart ventricle from the first anchor to the portion of the ventricular heart wall of the heart ventricle between the apex and the basal portion of the ventricular wall can comprise extending the portion of the tether within the left heart ventricle from the first anchor coupled to the mitral valve annulus. Positioning the second anchor over the externally oriented surface of the portion of the ventricular heart wall can comprise positioning the second anchor over an externally oriented surface of a portion of a ventricular heart wall of the left heart ventricle. In some instances, coupling the tether to the second anchor while the second anchor is over the externally oriented surface of the ventricular heart wall can comprise coupling the tether to the second anchor while the second anchor is over the externally oriented surface of the ventricular heart wall of the left heart ventricle.

In some instances, coupling the first anchor to the annulus of the heart valve can comprise coupling the first anchor to a portion of the mitral valve annulus adjacent to a posterior leaflet. Alternatively, coupling the first anchor to the annulus of the heart valve can comprise coupling the first anchor to a portion of the mitral valve annulus adjacent to an anterior leaflet. For example, extending the portion of the tether from the first anchor to the portion of the ventricular heart wall can comprise extending the tether from the first anchor to a portion of the ventricular heart wall adjacent to anterolateral papillary muscle.

As described herein, a tether system can comprise an elongate tube configured to receive at least a portion of the portion of the tether extending from the first anchor to the second anchor, including at least a portion of the tether configured to be positioned within the ventricle. In some instances, the tether system deployment process can involve advancing an elongate tube along at least a portion of the tether within the heart ventricle. The elongate tube can comprise a central lumen configured to receive at least a portion of the tether that is disposed within the heart ventricle. For example, the elongate tube can be advanced along at least a portion of the tether disposed within the heart ventricle, after the first anchor is deployed to its target site and the tether is extended from the first anchor. In some instances, the elongate tube can be configured to extend along an entire or substantially entire length of the tether disposed within the heart ventricle. For example, the tether system deployment process can involve advancing an elongate tube along the entire or substantially entire portion of the tether that is disposed within the heart ventricle.

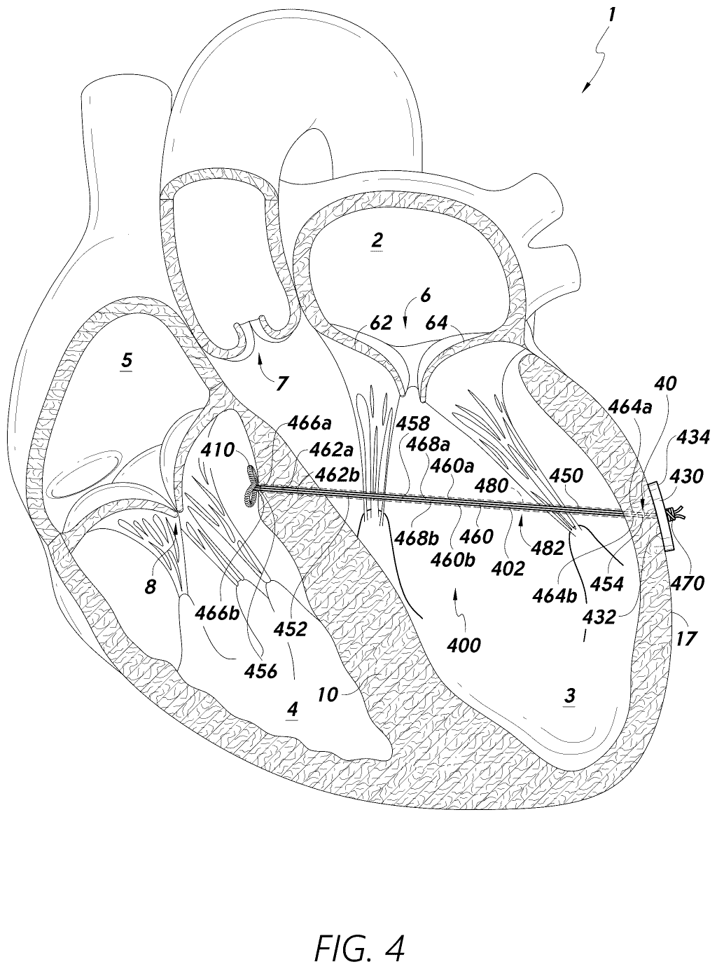

FIGS. 4, 5 and 6 show examples of tether systems 400, 500, 600 configured to couple a septal wall 10 of a heart 1 and a ventricular heart wall 17. Each of the tether systems 400, 500, 600 can comprise a first anchor 410, 510, 610 configured to be coupled to a portion of the septal wall 10 and a second anchor 430, 530, 630 configured to be coupled to a portion of the ventricular heart wall 17. A tether 450, 550, 650 can extend between the respective first anchors 410, 510, 610 and second anchors 430, 530, 630. Each tether 450, 550, 650 can be configured to couple the respective first anchors 410, 510, 610 and second anchors 430, 530, 630 to one another. For example, each of the tethers 450, 550, 650 can comprise a portion configured to be disposed through the heart ventricle between the first anchors 410, 510, 510 and second anchors 430, 530, 630. Coupling the ventricular heart wall 17 to the septal wall 10 can be configured to pull the ventricular heart wall 17 inwards and/or upwards. The portion of the ventricular heart wall 17 can be pulled toward the portion of the septal wall 10. For example, locations on the septal wall 10 to which the first anchors 410, 510, 610 are coupled and/or locations on the ventricular heart wall 17 to which the second anchors 430, 530, 630 are coupled can be selected such that desired relocation of the target portion of the ventricular heart wall 17 inwards and/or upwards can be achieved. In some instances, the first anchors 410, 510, 610 can be configured to be coupled to a basal portion of the septal wall 10. In some instances, the first anchors 410, 510, 610 can be configured to be coupled to a mid-portion of the septal wall 10. In some instances, the second anchors 430, 530, 630 can be configured to be coupled to a mid-portion of the ventricular heart wall 17.

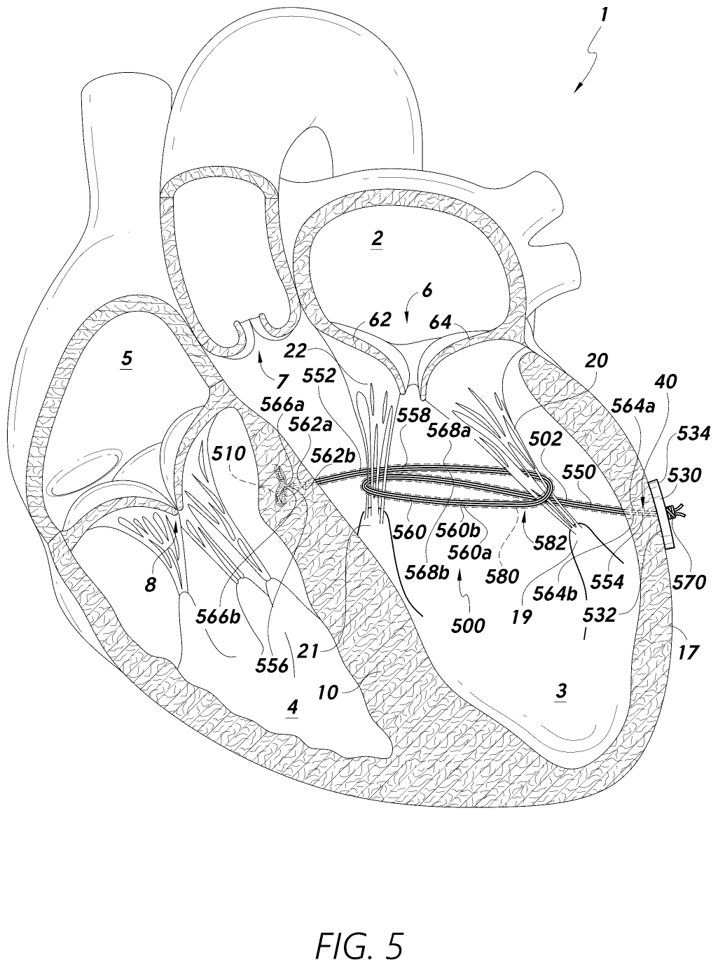

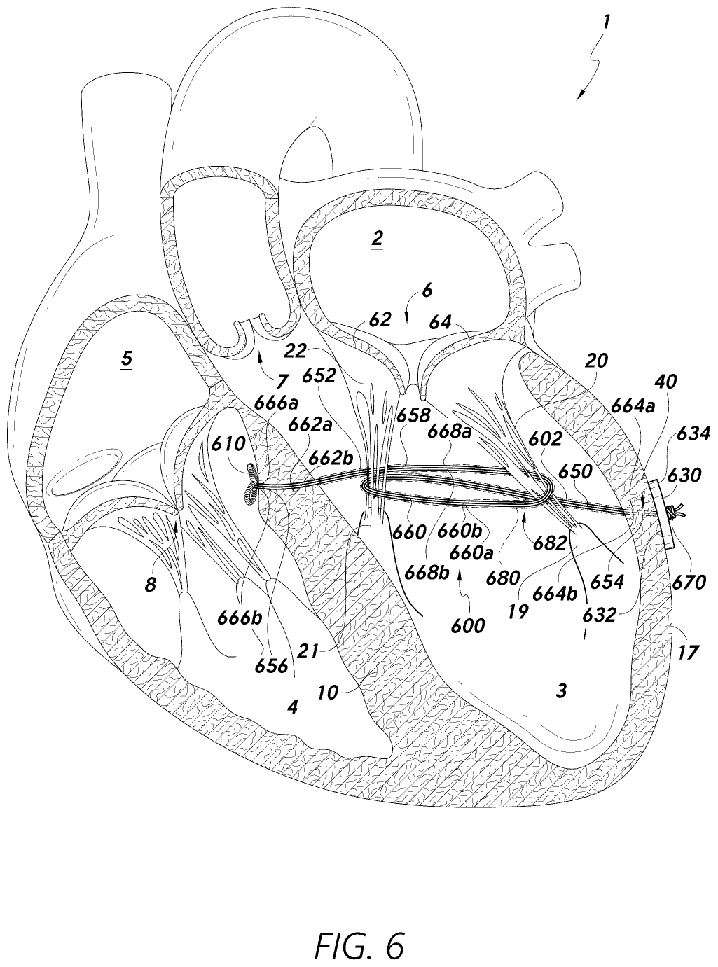

Each of the tethers 450, 550, 650 can comprise a first distal portion 452, 552, 652 and a second distal portion 454, 554, 654. The first distal portions 452, 552, 652 can be configured to be disposed through at least a portion of the septal wall 10 such that the tethers 450, 550, 650 can be coupled to the first anchors 410, 510, 610. For example, a first distal end 456, 556, 656 of the tether 450, 550, 650 can be coupled to the first anchor 410, 510, 610. As described in further detail herein, in some instances, a first anchor can be configured to be disposed over a surface of a septal wall. Alternatively, a first anchor can be at least partially, including completely, embedded within a septal wall. FIGS. 4 and 6 show the first anchors 410, 610 disposed over and/or in contact with a surface of the septal wall 10 having an orientation opposite relative to that oriented toward the heart ventricle through which the tethers 450, 650 extend. The first anchors 410, 510 can be configured to be disposed within a heart ventricle different from that within which the portion of the tethers 450, 650 are configured to be disposed. The first anchors 410, 610 are shown in FIGS. 4 and 6 as being disposed over and/or in contact with a surface of the septal wall 10 that is oriented toward a right heart ventricle 4. The first distal portions 452, 652 can comprise at least a portion configured to be disposed through an entire thickness of the septal wall 10 between the right and left ventricles 4, 3 such that the tethers 450, 650 can be coupled to the first anchors 410, 610. The first distal portion 152, 252, such as a first distal end 156, 256, of the tether 150, 250 can be coupled to the first anchor 110, 210. FIG. 5 shows the first anchor 510 embedded within the septal wall 10. The first distal portion 552 of the tether 550 can be configured to be disposed through only a portion of a thickness of the septal wall 10. A medial portion 458, 558, 658 of the tethers 450, 550, 650 can be disposed within the left ventricle 3.

Each of the second anchors 430, 530, 630 can be configured to be disposed over an externally oriented surface of the portion of the ventricular heart wall 17. The second distal portions 454, 554, 654 can comprise at least a portion configured to extend through the portion of the ventricular heart wall 17 to couple to the second anchors 430, 530, 630. FIGS. 4, 5 and 6 show the second anchors 430, 530, 630 disposed over an externally oriented surface of a portion of the ventricular heart wall 17 of the left ventricle 3. The second distal portions 454, 554, 654 can comprise at least a portion configured to extend through the portion of the ventricular heart wall 17 of the left ventricle 3 to couple to the second anchors 430, 530, 630. The tethers 450, 550, 650 can comprise a respective portion configured to extend through each of at least a portion of the septal wall 10, through the left heart ventricle 3 and through the ventricular heart wall 17 of the left heart ventricle 3.

FIG. 4 shows the tether 450 being configured to extend directly between the first anchor 410 and the second anchor 430. For example, the tether 450 can extend between the first anchor 410 and the second anchor 430 without forming any loops. The tether 450 can extend between the portion of the septal wall 10 and the portion of the ventricular heart wall 17 of the left ventricle 3 without forming any loops. FIGS. 5 and 6 show the tethers 550, 650 comprising a portion configured to form one or more loops within the heart ventricle around papillary muscles of the heart ventricle and/or chordae tendineae coupled to the papillary muscles. The tethers 550, 650 can comprise a portion configured to form a loop around respective portions of the papillary muscles and chordae tendineae in the heart ventricle. Medial portions 558, 658 of the tethers 550, 650 disposed within the left ventricle 3 can be configured to form a loop around one or more of the papillary muscles 19, 21 in the left ventricle 3 and/or chordae tendineae 20, 22 coupled to the papillary muscles 19, 21. For example, medial portions 558, 658 of the tethers 550, 650 can be configured to form a loop around both a posteromedial papillary muscle 21 or chordae tendineae 22 coupled to the posteromedial papillary muscle 21, and an anterolateral papillary muscle 19 or chordae tendineae 20 coupled to the anterolateral papillary muscle 19.

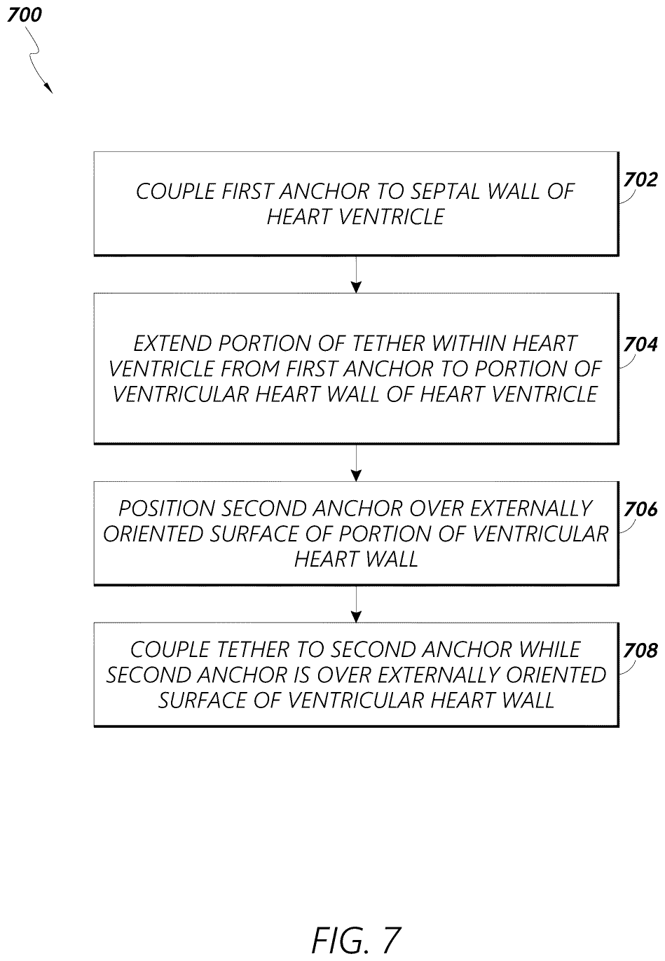

FIG. 7 is a process flow diagram showing an example of a process 700 for deploying a tether system as described herein, including one or more of the tether systems 400, 500, 600 described with reference to FIGS. 4, 5 and 6. In block 702, the process can involve coupling a first anchor to a septal wall of a heart ventricle. In block 704, the process can involve extending a portion of a tether within the heart ventricle from the first anchor to a portion of a ventricular heart wall of the heart ventricle. In block 706, the process can involve positioning a second anchor over an externally oriented surface of the portion of the ventricular heart wall. In block 708, the process can involve coupling the tether to the second anchor while the second anchor is over the externally oriented surface of the ventricular heart wall.

In some instances, coupling the first anchor to the septal wall can comprise embedding the first anchor within the septal wall. Extending the portion of the tether from the first anchor to the portion of the ventricular heart wall of the heart ventricle can comprise extending a portion of the tether through a portion of the septal wall. Alternatively, the first anchor may not be within the septal wall. In some instances, the first anchor can be disposed over a surface of the septal wall. For example, coupling the first anchor to the septal wall can comprise positioning the first anchor over a surface of the septal wall having an opposing orientation relative to that oriented toward the heart ventricle. The first anchor can be disposed in a different heart ventricle than that in which the tether extends. For example, the first anchor can be disposed over and be in contact with a surface of the septal wall oriented toward the different heart ventricle. Extending the portion of the tether from the first anchor to the portion of the ventricular heart wall of the heart ventricle can comprise extending a portion of the tether through an entire thickness of the septal wall, such as from the surface over which the first anchor is disposed to the surface of the septal wall oriented toward the heart ventricle within which the tether extends.

In some instances, coupling the first anchor to the septal wall can comprise coupling the first anchor to a basal portion of the septal wall. In some instances, coupling the first anchor to the septal wall can comprise coupling the first anchor to a mid-portion of the septal wall. In some instances, the second anchor can be disposed over an externally oriented surface of a mid-portion of the ventricular heart wall. For example, extending the portion of the tether from the first anchor to the portion of the ventricular heart wall of the heart ventricle can comprise extending the portion of the tether from the first anchor to a mid-portion of the ventricular heart wall. In some instances, extending the portion of the tether from the first anchor to the portion of the ventricular heart wall of the heart ventricle can comprise extending the portion of the tether from the first anchor coupled to a basal portion of the septal wall to a mid-portion of the ventricular heart wall. In some instances, extending the portion of the tether from the first anchor to the portion of the ventricular heart wall of the heart ventricle can comprise extending the portion of the tether from the first anchor coupled to a mid-portion of the septal wall to a mid-portion of the ventricular heart wall.

In some instances, extending the portion of the tether from the first anchor to the portion of the ventricular heart wall of the heart ventricle can comprise extending the portion of the tether directly from the first anchor to the second anchor. For example, the tether can extend between the first and second anchors without making any loops. Alternatively, the portion of the tether extending between the first and second anchors can form one or more loops. In some instances, extending the portion of the tether from the first anchor to the portion of the ventricular heart wall of the heart ventricle can comprise looping the tether around papillary muscles and/or chordae tendineae coupled to the papillary muscles. For example, a portion of the tether can form one or more loops around both papillary muscles and/or chordae tendineae coupled to the papillary muscles of the heart ventricle.

In some instances, the heart ventricle can be a left heart ventricle. Extending a portion of a tether within the heart ventricle from the first anchor to a portion of a ventricular heart wall of the heart ventricle can comprise extending a portion of a tether within the left heart ventricle from the first anchor to a portion of a ventricular heart wall of the left heart ventricle. In some instances, positioning a second anchor over an externally oriented surface of the portion of the ventricular heart wall can comprise positioning a second anchor over an externally oriented surface of the portion of the ventricular heart wall of the left heart ventricle. In some instances, coupling the tether to the second anchor while the second anchor is over the externally oriented surface of the ventricular heart wall can comprise coupling the tether to the second anchor while the second anchor is over the externally oriented surface of the ventricular heart wall of the left heart ventricle. Extending the portion of the tether from the first anchor to the portion of the ventricular heart wall of the heart ventricle can comprise looping the tether around both a posteromedial papillary muscle or chordae tendineae coupled to the posteromedial papillary muscle, and an anterolateral papillary muscle or chordae tendineae coupled to the anterolateral papillary muscle.

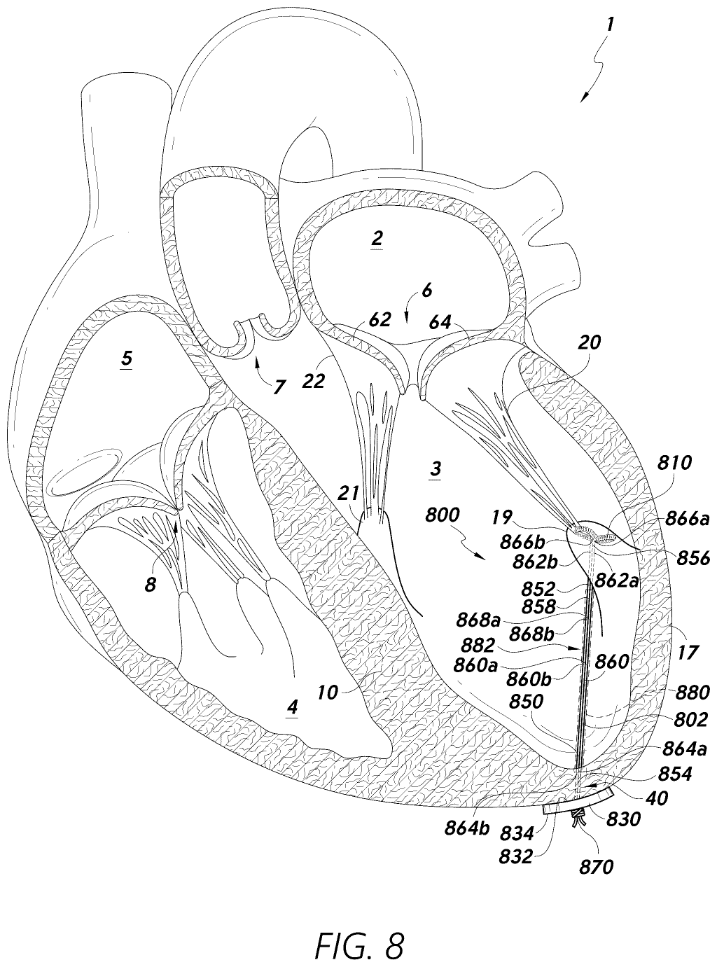

FIG. 8 shows a tether system 800 deployed in a heart 1 and configured to couple a papillary muscle of a heart ventricle, such as a left heart ventricle 3, to a ventricular heart wall 17 of the heart ventricle 1. The tether system 800 can comprise a first anchor 810 configured to be coupled to the papillary muscle of the heart ventricle. A second anchor 830 can be configured to be coupled to the ventricular heart wall 17 of the heart ventricle. A tether 850 can be configured to couple the first anchor 810 and the second anchor 830 to one another. For example, the tether 850 can comprise a portion, such as a medial portion 858, configured to extend within the heart ventricle between the first anchor 810 and the second anchor 830. A first distal portion 852 of the tether 850 can be coupled to the first anchor 810. A second distal portion 854 of the tether 850 can be coupled to the second anchor 830. The first anchor 810 can be configured to be disposed over a surface of the papillary muscle. The second anchor 830 can be configured to be positioned over an externally oriented surface the ventricular heart wall 17. For example, at least a portion of the first distal portion 852 of the tether 850 can be configured to extend through a portion of the papillary muscle such that the first distal portion 852, such as a first distal end 856, can be coupled to the first anchor 810. At least a portion of the second distal portion 854 of the tether 850 can be configured to extend through a portion of the ventricular heart wall 17 such that the second distal portion 854 can be coupled to the second anchor 830.

In some instances, the second anchor 830 can be configured to be positioned over an externally oriented surface of an apical portion, including an apex, of the ventricular heart wall 17 of the heart ventricle. For example, at least a portion of the second distal portion 854 of the tether 850 can be configured to extend through the apical portion of the ventricular heart wall 17 such that the second distal portion 854 can be coupled to the second anchor 830. In some instances, at least a portion of the second distal portion 854 of the tether 850 can be configured to extend through the apex of the ventricular heart wall 17 such that the second distal portion 854 can be coupled to the second anchor 830. Coupling the papillary muscle to an apical portion of the ventricular heart wall 17 can be configured to pull the papillary muscle and/or a portion of the ventricular heart wall 17 adjacent to papillary muscle inward. For example, the papillary muscle and/or a portion of the ventricular heart wall 17 from which the papillary muscle extends can be pulled inward.

As described herein, in some instances, the heart ventricle can be the left ventricle 3. The first anchor 810 can be configured to be coupled to a papillary muscle of the left ventricle 3, including an anterolateral papillary muscle 19 of the left ventricle 3. For example, the first anchor 810 can be disposed over a surface of the anterolateral papillary muscle 19. The second anchor 830 can be configured to be disposed over an externally oriented surface of an apical portion, including an apex, of the left ventricular heart wall. At least a portion of the first distal portion 852 of the tether 850 can be configured to extend through the anterolateral papillary muscle 19 such that the first distal portion 852 can be coupled to the first anchor 810. At least a portion of the second distal portion 854 of the tether 850 can be configured to extend through the apical portion of the left ventricular heart wall, such as the apex, such that the second distal portion 854 can be coupled to the second anchor 830.

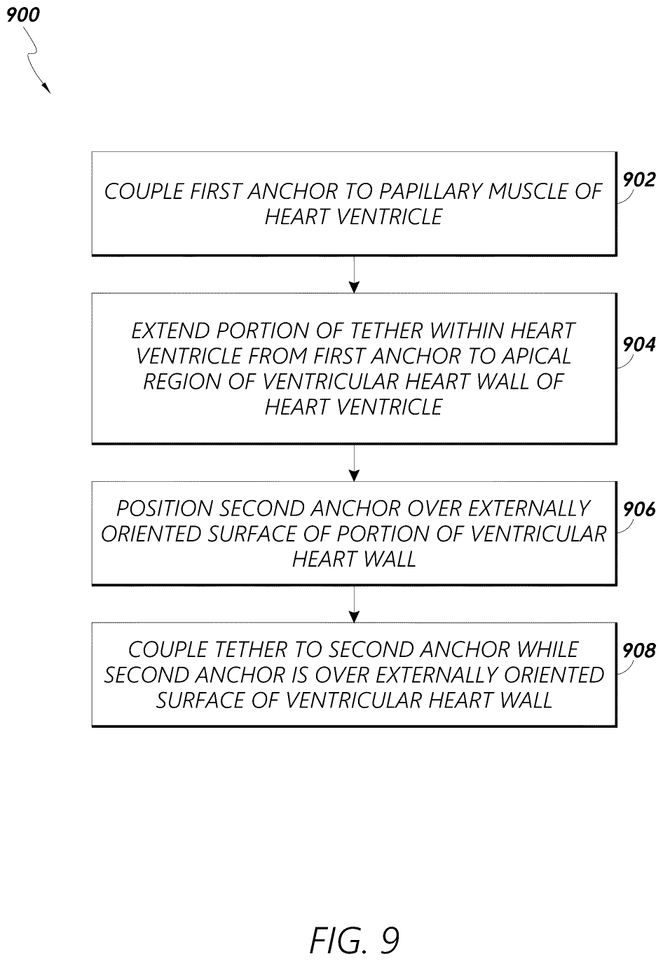

FIG. 9 is a process flow diagram showing an example of a process 900 for deploying a tether system as described herein, such as the tether system 800 described with reference to FIG. 8. In block 902, the process can involve coupling a first anchor to a papillary muscle of a heart ventricle. In block 904, the process can involve extending a portion of a tether within the heart ventricle from the first anchor to an apical portion of a ventricular heart wall of the heart ventricle. In block 906, the process can involve positioning a second anchor over an externally oriented surface of the apical portion of the ventricular heart wall. In block 908, the process can involve coupling the tether to the second anchor while the second anchor is over the externally oriented surface of the apical portion of the ventricular heart wall.

In some instances, the heart ventricle can be the left heart ventricle. For example, coupling the first anchor to the papillary muscle can comprise coupling the first anchor to a papillary muscle of the left heart ventricle. In some instances, coupling the first anchor to the papillary muscle can comprise coupling the first anchor to an anterolateral papillary muscle of the left heart ventricle. For example, extending the portion of the tether within the heart ventricle from the first anchor to the apical portion of the ventricular heart wall can comprise extending the portion of the tether within the heart ventricle from the first anchor coupled to the anterolateral papillary muscle to the apical portion of the left heart ventricle.

In some instances, the papillary muscle can be coupled to an apex of the heart ventricle, such as the left heart ventricle. For example, extending the portion of the tether within the heart ventricle from the first anchor to the apical portion of the ventricular heart wall can comprise extending the portion of the tether within the heart ventricle from the first anchor to an apex of the ventricular heart wall. In some instances, extending the portion of the tether within the heart ventricle from the first anchor to the apical portion of the ventricular heart wall can comprise extending the portion of the tether within the heart ventricle from the first anchor coupled to the anterolateral papillary muscle of the left heart ventricle to an apex of the ventricular heart wall of the left heart ventricle.

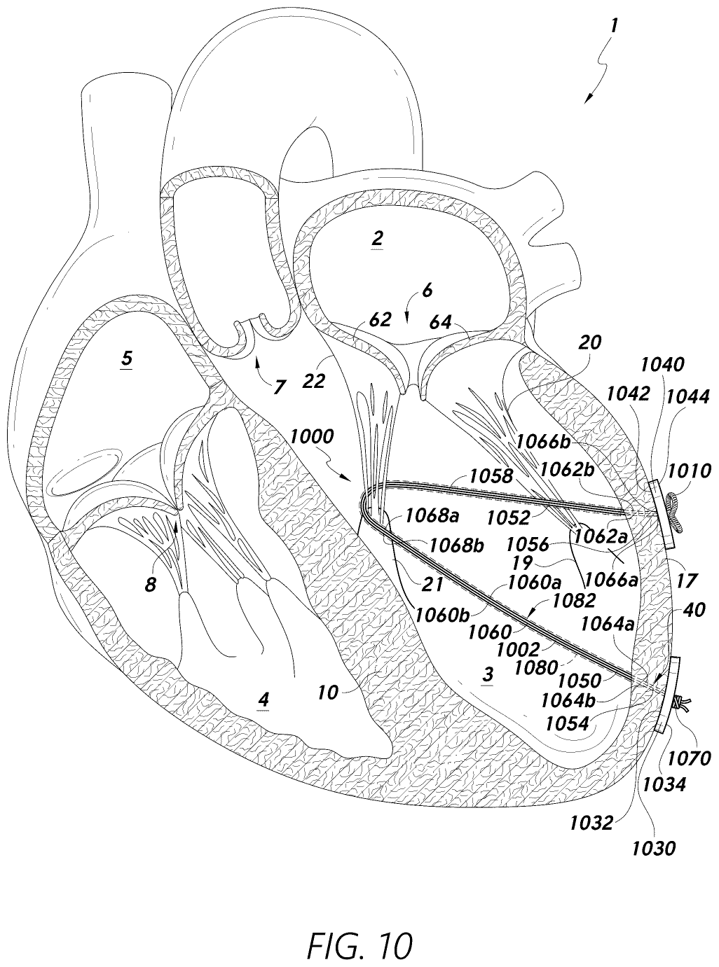

FIG. 10 shows a tether system 1000 deployed in a heart 1 and configured to couple a first portion of a ventricular heart wall 17 and a second portion of the ventricular heart wall 17 of a heart ventricle to one another via a papillary muscle of the heart ventricle. The tether system 1000 can comprise a first anchor 1010 configured to be coupled to the first portion of the ventricular heart wall 17 and a second anchor 1030 configured to be coupled to the second portion of the ventricular heart wall 17. A tether 1050 can be configured to couple the first anchor 1010 and the second anchor 1030 to one another. The tether 1050 can comprise a portion configured to form at least a partial loop around a papillary muscle of the heart ventricle or chordae tendineae coupled to the papillary muscle. For example, the tether 1050 can comprise a medial portion 1058 configured to extend within the heart ventricle between the first and second anchors 1010, 1030, and the portion of the tether 1050 configured to be disposed within the ventricle configured to form at least a partial loop around a papillary muscle of the heart ventricle or chordae tendineae coupled to the papillary muscle. The tether system 1000 can be configured to pull the portion of the ventricular heart wall inward and toward the papillary muscle.

The first anchor 1010 can be configured to be disposed over an externally oriented surface of a first portion of the ventricular heart wall 17. The second anchor 1030 can be configured to be disposed over an externally oriented surface of a second portion of the ventricular heart wall 17. The tether 1050 can be configured to extend between the first anchor 1010 and the second anchor 1030 while making at least a partial loop around the papillary muscle or chordae tendineae coupled to the papillary muscle. The tether 1050 can comprise a first distal portion 1052 configured to be coupled to the first anchor 1010 and a second distal portion 1054 of the tether 1050 can be coupled to the second anchor 1030. For example, at least a portion of the first distal portion 1052 of the tether 1050 can be configured to extend through the first portion of the ventricular heart wall 17 such that the first distal portion 1052, such as a first distal end 1056, can be coupled to the first anchor 1010 disposed over the externally oriented surface of the first portion of the ventricular heart wall 17. At least a portion of the second distal portion 1054 of the tether 1050 can be configured to extend through the second portion of the ventricular heart wall 17 such that the second distal portion 1054 can be coupled to the second anchor 1030 disposed over the externally oriented surface of the second portion of the ventricular heart wall 17.

The first portion of the ventricular heart wall 17 can be above the second portion of the ventricular heart wall 17. In some instances, at least one of the first portion and the second portion are in a mid-portion of the ventricular heart wall 17. In some instances, at least one of the first portion and the second portion are in an apical portion of the ventricular heart wall 17. For example, the first portion of the ventricular heart wall 17 can be in a mid-portion while the second portion of the ventricular heart wall 17 can be in the apical portion. In some instances, both the first and second portions of the ventricular heart wall 17 can be in the mid-portion. In some instances, both the first and second portions of the ventricular heart wall 17 can be in the apical portion.

In some instances, the heart ventricle can be the left ventricle 3. The first anchor 1010 can be configured to be disposed over an externally oriented surface of a first portion of the ventricular heart wall 17 of the left ventricle 3. The second anchor 1030 can be configured to be disposed over an externally oriented surface of a second portion of the ventricular heart wall 17 of the left ventricle 3. The tether 1050 can be configured to extend between the first anchor 1010 and the second anchor 1030 while making at least a partial loop around a papillary muscle or chordae tendineae coupled to the papillary muscle of the left ventricle 3, including the posteromedial papillary muscle 21 of the left ventricle 3. For example, a portion of the tether 1050 can form a partial loop around the posteromedial papillary muscle 21 or chordae tendineae 22 coupled to the posteromedial papillary muscle 21 of the left ventricle 3.

In some instances, a pad 1040 can be deployed over and/or in contact with the first portion of the ventricular heart wall 17 to facilitate maintaining the first anchor 1010 at the desired location. For example, a first surface 1042 of the pad 1040 can be configured to be oriented toward and/or be in contact with the ventricular heart wall 17 and a second surface 1044 of the pad 1040 can be configured to be oriented away from the ventricular heart wall 17. The first anchor 1010 can be disposed over and/or in contact with the second surface 1044. The first distal portion 1052 of the tether 1050 can extend through the pad 1040 to couple to the first anchor 1010.

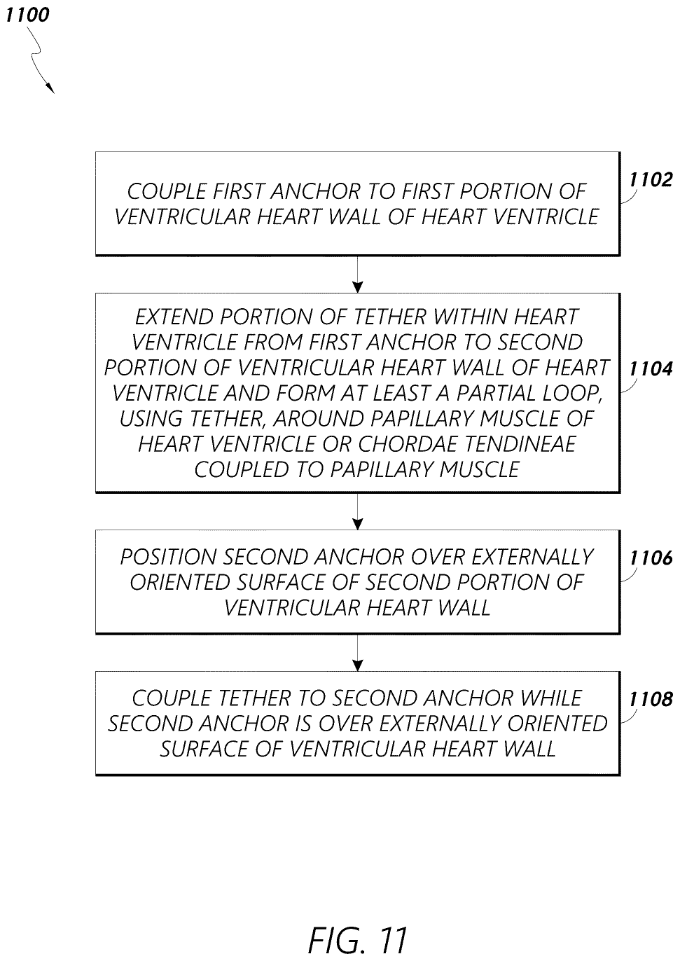

FIG. 11 is a process flow diagram of an example of a process 1100 for deploying a tether system as described herein, including the tether system 1000 described with reference to FIG. 10. In block 1102, the process can involve coupling a first anchor to a first portion of a ventricular heart wall of a heart ventricle. In block 1104, the process can involve extending a portion of a tether within the heart ventricle from the first anchor to a second portion of the ventricular heart wall of the heart ventricle and forming at least a partial loop, using the tether, around a papillary muscle of the heart ventricle or chordae tendineae coupled to the papillary muscle. In block 1106, the process can involve positioning a second anchor over an externally oriented surface of the second portion of the ventricular heart wall. In block 1108, the process can involve coupling the tether to the second anchor while the second anchor is over the externally oriented surface of the ventricular heart wall, such as while the second anchor is over the second portion of the ventricular heart wall.

The first portion of the ventricular heart wall can be at a higher position on the ventricular wall than the second portion of the ventricular heart wall. For example, extending the portion of the tether within the heart ventricle from the first anchor to the second portion of the ventricular heart wall can comprise extending the portion of the tether within the heart ventricle from the first anchor to a second portion of the ventricular heart wall that is lower on the ventricular heart wall than the first portion.

In some instances, both the first portion and the second portion of the ventricular heart wall can be at respective positions on a mid-portion of the ventricular heart wall. For example, coupling the first anchor to the first portion of the ventricular heart wall can comprise coupling the first anchor to a first mid-portion of the ventricular heart wall. In some instances, extending the portion of the tether configured to be within the heart ventricle from the first anchor to the second portion of the ventricular heart wall can comprise extending the portion of the tether within the heart ventricle from the first anchor to a second mid-portion of the ventricular heart wall. As described herein, the first portion of the ventricular heart wall can be at a higher position on the ventricular wall than the second portion of the ventricular heart wall. In some instances, extending the portion of the tether configured to be within the heart ventricle from the first anchor to the second portion of the ventricular heart wall can comprise extending the portion of the tether within the heart ventricle from the first anchor to a second mid-portion of the ventricular heart wall that is at a position lower than the first portion of the ventricular heart wall.

In some instances, the first portion of the ventricular heart wall can be on a mid-portion of the ventricular heart wall while the second portion of the ventricular heart wall can be on an apical portion of the ventricular heart wall. For example, coupling the first anchor to the first portion of the ventricular heart wall can comprise coupling the first anchor to a mid-portion of the ventricular heart wall. Extending the portion of the tether configured to be within the heart ventricle from the first anchor to the second portion of the ventricular heart wall can comprises extending the portion of the tether within the heart ventricle from the first anchor to an apical portion of the ventricular heart wall.

In some instances, both the first portion and the second portion of the ventricular heart wall can be at respective positions on an apical portion of the ventricular heart wall. For example, coupling the first anchor to the first portion of the ventricular heart wall can comprise coupling the first anchor to a first apical portion of the ventricular heart wall. In some instances, extending the portion of the tether configured to be within the heart ventricle from the first anchor to the second portion of the ventricular heart wall can comprise extending the portion of the tether within the heart ventricle from the first anchor to a second apical portion of the ventricular heart wall. As described herein, the first portion of the ventricular heart wall can be at a higher position on the ventricular wall than the second portion of the ventricular heart wall. In some instances, extending the portion of the tether configured to be within the heart ventricle from the first anchor to the second portion of the ventricular heart wall can comprise extending the portion of the tether within the heart ventricle from the first anchor to a second apical portion of the ventricular heart wall that is at a position lower than the first portion of the ventricular heart wall.

In some instances, forming at least a partial loop, using the tether, around the papillary muscle can comprise forming at least a partial loop, using the tether, around a posteromedial papillary muscle or chordae tendineae coupled to the posteromedial papillary muscle. For example, a partial loop can be formed around the posteromedial papillary muscle or chordae tendineae coupled to the posteromedial papillary muscle.

It will be understood that the tether systems 400, 500, 600, 800, 1000 described with reference to FIGS. 4, 5, 6, 8 and 10 can have one or more features of the tether systems 100, 200 described with reference to FIGS. 1 and 2. For example, each of the second anchors 430, 530, 630, 830, 1030 can comprise a first surface 432, 532, 632, 832, 1032 configured to be oriented toward the ventricular heart wall and a second surface 434, 534, 634, 834,1034 configured to be oriented away from the ventricular heart wall. The first surface 432, 532, 632, 832, 1032 can be configured to be over and in contact with the ventricular heart wall. In some instances, the second distal portion 454, 554, 654, 854, 1054 of the tether 450, 550, 650, 850, 1050 can be configured to extend through the respective ventricular heart wall and the second anchor 430, 530, 630, 830, 1030 such that a knot 470, 570, 670, 870, 1070 can be formed over the second surface 434, 534, 634, 834,1034 of the respective second anchor 430, 530, 630, 830, 1030 to couple the tether 450, 550, 650, 850, 1050 to the second anchor 430, 530, 630, 830, 1030.

In some instances, the first anchor 410, 510, 610, 810, 1010 and the tether 450, 550, 650, 850, 1050 can be an integral piece. In some instances, the first anchor 410, 510, 610, 810, 1010 and the tether 450, 550, 650, 850, 1050 can be formed using the same material. The first anchor 410, 510, 610, 810, 1010 and the tether 450, 550, 650, 850, 1050 can each comprise a respective portion of a surgical cord and/or suture. For example, a portion of a suture 402, 502, 602, 802, 1002 can form the first anchor 410, 510, 610, 810, 1010. Another portion of the suture 402, 502, 602, 802, 1002 can form the tether 450, 550, 650, 850, 1050. As described in further detail herein, in some instances, the first anchor 410, 510, 610, 810, 1010 can comprise a bulky-knot. For example, a portion of the suture 402, 502, 602, 802, 1002 can be deployed through the portion of the septal wall, papillary muscle or ventricular heart wall to form the first anchor 410, 510, 610, 810, 1010. Another portion of the suture 402, 502, 602, 802, 1002 can form the tether 450, 550, 650, 850, 1050, for example extending through the septal wall, papillary muscle or ventricular heart wall, the ventricle, and another portion of the ventricular wall, such that suture 402, 502, 602, 802, 1002 can be coupled to the second anchor 430, 530, 630, 830, 1030.

In some instances, each of the tethers 450, 550, 650, 850, 1050 can comprise respective tether tails 460, 560, 660, 860, 1060. For example, the tethers 450, 550, 650, 850, 1050 can each comprise a pair of tether tails 460a, 460b, 560a, 560b, 660a, 66ob, 860a, 860b, 1060a, 1060b, each comprising a respective first distal portion 462a, 462b, 562a, 562b, 662a, 662b, 862a, 862b, 1062a, 1062b configured to extend through the portion of the septal wall, papillary muscle or ventricular heart wall and a second distal portion 464a, 464b, 564a, 564b, 664a, 664b, 864a, 864b, 1064a, 1064b configured to extend through another portion of the ventricular heart wall and couple to the respective second anchors 430, 530, 630, 830, 1030. For example, the first distal portion 462a, 462b, 562a, 562b, 662a, 662b, 862a, 862b, 1062a, 1062b, including the first distal end 466a, 466b, 566a, 566b, 666a, 666b, 866a, 866b, 1066a, 1066b, can be configured to the first anchor 410, 510, 610, 810, 1010. A medial portion 468a, 468b, 568a, 568b, 668a, 668b, 868a, 868b, 1068a, 1068b can be configured to extend through the ventricle. The second distal portions 464a, 464b, 564a, 564b, 664a, 664b, 864a, 864b, 1064a, 1064b can be configured to extend through the respective second anchors 430, 530, 630, 830, 1030 and form the knot 470, 570, 670, 870, 1070 for coupling the tethers 450, 550, 650, 850, 1050 to the second anchors 430, 530, 630, 830, 1030.

In some instances, the tether systems 400, 500, 600, 800, 1000 can optionally comprise an elongate tube 480, 580, 680, 880, 1080 comprising a lumen 482, 582, 682, 882, 1082 configured to receive at least a portion of the tether 450, 550, 650, 850, 1050 extending within the heart ventricle. The elongate tube 480, 580, 680, 880, 1080 can be configured to prevent or reduce damage to heart tissue due to contact between the tether 450, 550, 650, 850, 1050 and the heart tissue. For example, a process for deploying the tether system 400, 500, 600, 800, 1000 can comprise advancing the elongate tube 480, 580, 680, 880, 1080 along at least a portion of a respective tether 450, 550, 650, 850, 1050 within the heart ventricle. In some instances, the second distal portion 454, 554, 654, 854, 1054 the respective tether 450, 550, 650, 850, 1050 can be positioned into the lumen 482, 582, 682, 882, 1082 of the elongate tube 480, 580, 680, 880, 1080 such that the elongate tube elongate tube 480, 580, 680, 880, 1080 can be advanced along the respective tether 450, 550, 650, 850, 1050.

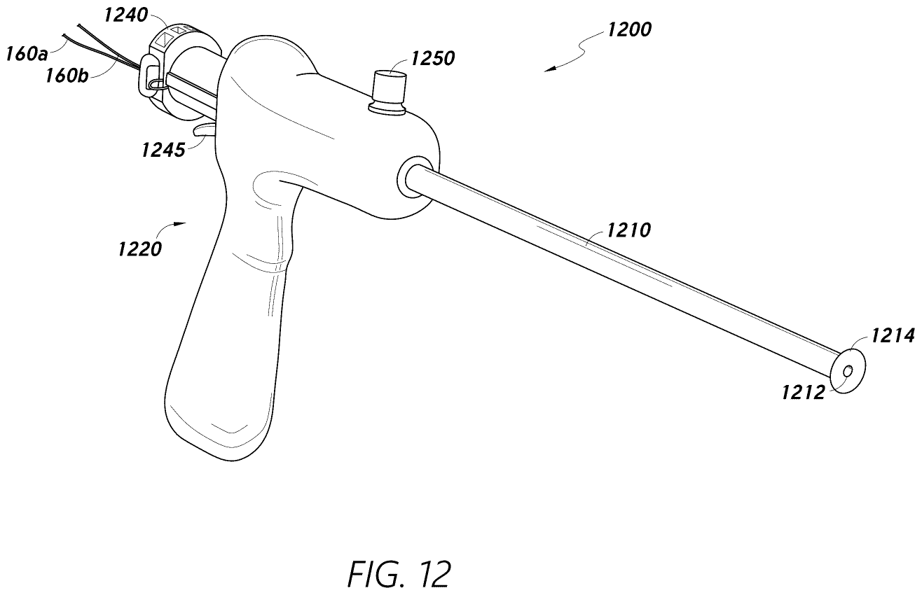

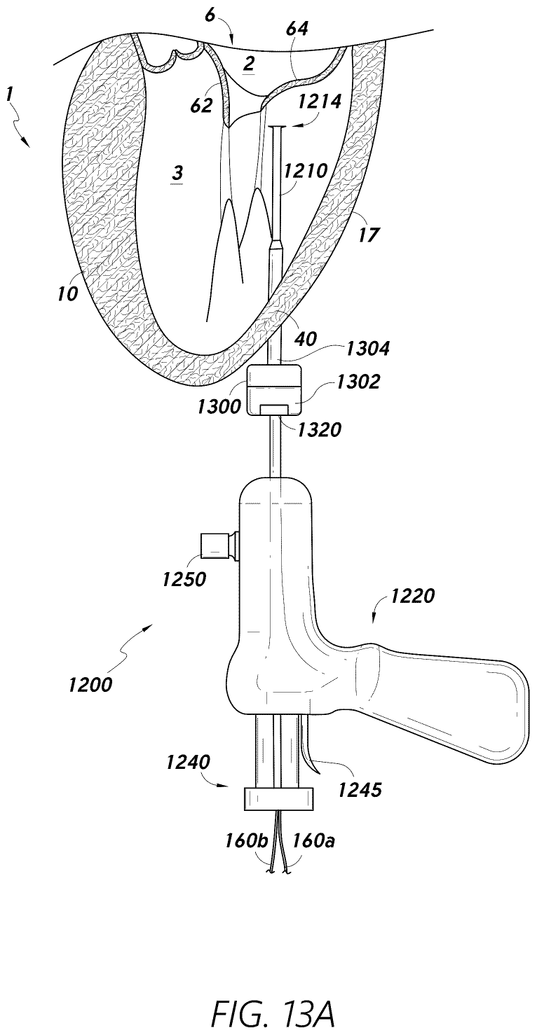

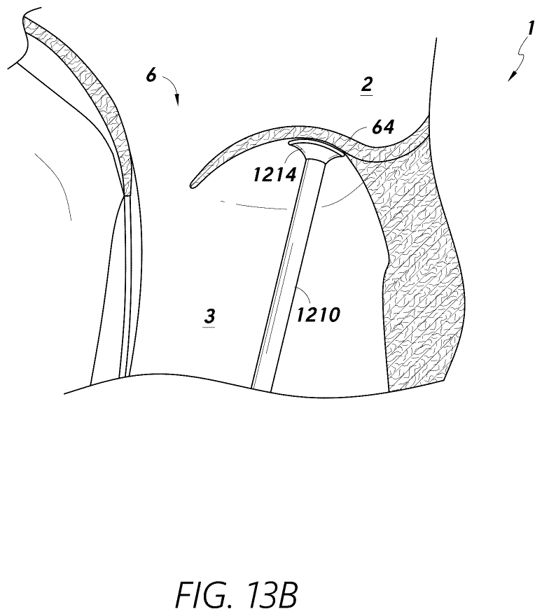

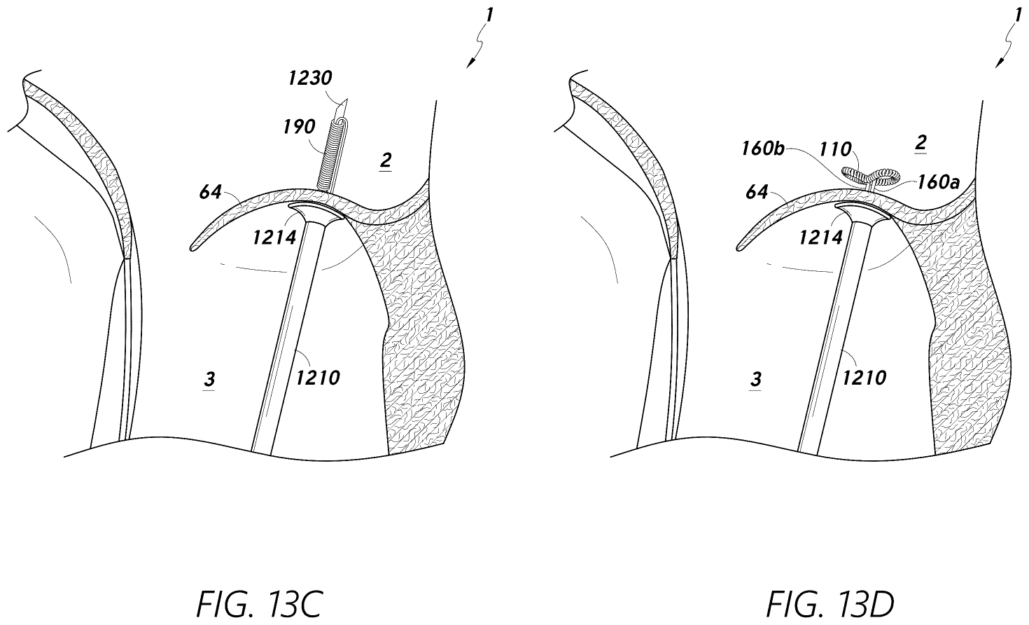

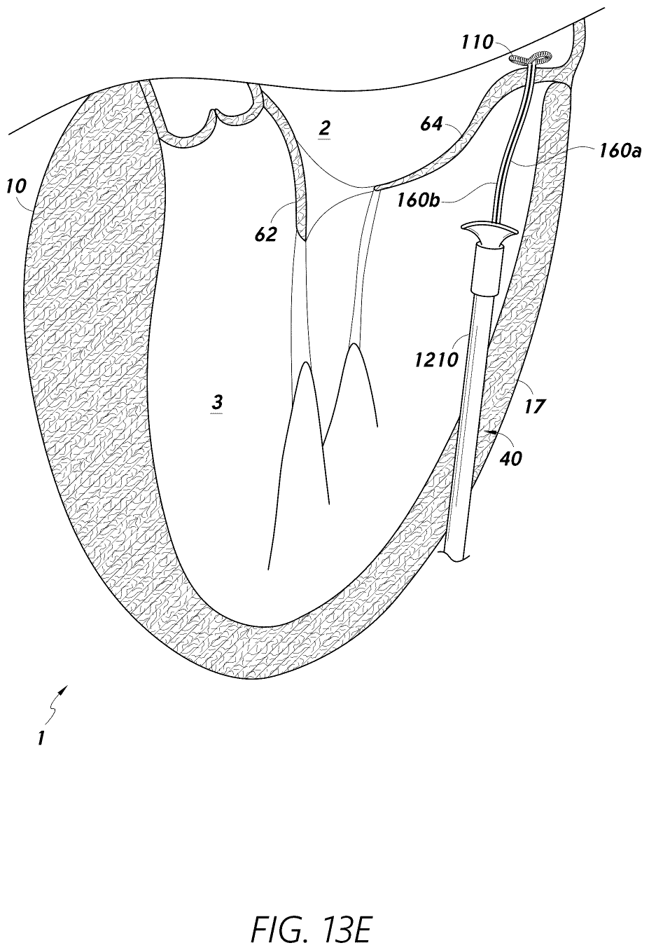

FIG. 12 is a perspective view of a tether delivery system 1200 in accordance with one or more examples. The tether delivery system 1200 may be used in the deployment of one or more tether systems as described herein. For example, the tether delivery system 1200 can be used in the deployment of the tether system 100 described with reference to FIG. 1. FIGS. 13A-13E describe an example of a process for using the tether delivery system 1200 to deliver the first anchor 110 and tether 150 of the tether system 100 to a heart valve annulus. The tether delivery system 1200 may be utilized to deliver and anchor tissue anchors, such as suture-knot-type tissue anchors, to one or more portions of the heart, such as a heart valve annulus, septal wall, papillary muscle, and/or ventricular heart wall. Such procedures may be implemented on a beating heart.

The tether delivery system 1200 can include an elongate rigid tube 1210 forming at least one internal working lumen. Although described in certain examples and/or contexts as comprising an elongate rigid tube, it should be understood that tubes, shafts, lumens, conduits, and the like disclosed herein may be either rigid, at least partially rigid, at least flexible, and/or at least partially flexible. Therefore, any such component described herein, whether or not referred to as rigid herein, should be interpreted as possibly being at least partially flexible. In alternative instances, an articulating and/or steerable elongate tube can be used to facilitate navigation within the patient anatomy to facilitate deployment of the tether systems.

In addition to the elongate rigid tube 1210, the tether delivery system 1200 may include a plunger feature 1240, which may be used or actuated to manually deploy the tether. The tether delivery system 1200 may further include a plunger lock mechanism 1245, which may serve as a safety lock that locks the tether delivery system 1200 until ready for use or deployment of a tether as described herein. The plunger feature 1240 may have associated therewith a suture-release mechanism, which may be configured to lock in relative position a pair of tether tails 160a, 16ob associated with a pre-formed suture knot sutureform anchor (not shown) to be deployed. The tether delivery system 1200 may further comprise a flush port 1250, which may be used to de-air the lumen of the elongate rigid tube 1210. For example, heparinized saline flush, or the like, may be connected to the flush port 1250 using a female Luer fitting to de-air the tether delivery system 1200. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure.

The lumen of the elongate rigid tube 1210 may house a needle (not shown) that is wrapped at least in part with a pre-formed suture knot sutureform anchor, as described in detail herein. In some instances, the elongate rigid tube 1210 presents a relatively low profile. For example, the elongate rigid tube 1210 may have a diameter of about 3 millimeters (mm) or less (e.g., about 9 French (Fr) or less). The elongate rigid tube 1210 can be associated with an atraumatic tip 1214 feature. The atraumatic tip 1214 can be an echogenic positioner component, which may be used for deployment and/or positioning of the suture-type tissue anchor. The atraumatic tip 1214, disposed at the distal end of the elongate rigid tube 1210, may be configured to have deployed therefrom a wrapped pre-formed suture knot sutureform anchor. For example, deployment of the wrapped pre-formed suture knot sutureform anchor can comprise advancing the wrapped pre-formed suture knot sutureform anchor through a distal end opening 1212 of the elongate rigid tube 1210 while the atraumatic tip 1214 is in contact with target tissue, as described herein

The atraumatic tip 1214 may be referred to as an “end effector.” In addition to a pre-formed suture knot sutureform anchor and associated needle, the elongate rigid tube 1210 may house an elongated knot pusher tube (not shown; also referred to herein as a “pusher”), which may be actuated using the plunger 1240 in some examples. As described in further detail below, the atraumatic tip 1214 can provide a surface against which the target heart surface portion may be held to facilitate deployment of an anchor through the target heart surface portion.

The tether delivery system 1200 may be used to deliver a “bulky-knot” type tissue anchor to a target heart location, as described in greater detail below. The atraumatic tip 1214 can be coupled to the distal end portion of the elongate rigid tube 1210, wherein the proximal end portion of the elongate rigid tube 1210 may be coupled to a handle portion 1220 of the tether delivery system 1200, as shown. The atraumatic tip 1214 (e.g., end effector), can be placed in contact with a surface of the target heart location to facilitate deployment of the “bulky-knot” anchor. Generally, the elongate pusher (not shown) may be movably disposed within a lumen of the elongate rigid tube 1210 and coupled to a pusher hub (not shown) that is movably disposed within the handle 1220 and releasably coupled to the plunger 1240. A needle (not shown) carrying a pre-formed suture knot sutureform anchor can be movably disposed within a lumen of the pusher and coupled to a needle hub (not shown) that is also coupled to the plunger 1240. The “bulky-knot” anchor can be formed using the pre-formed suture knot sutureform anchor carried on the needle. The plunger 1240 can be used to actuate or move the needle and the pusher during deployment of the “bulky-knot” anchor (see, e.g., FIGS. 13C and 13D). The plunger 1240 can be movably disposed at least partially within the handle 1220. For example, the handle 1220 may define a lumen in which the plunger 1240 can be moved. During operation, the pusher may also move within the lumen of the handle 1220. The plunger lock 1245 can be used to prevent the plunger 1240 from moving within the handle 1220 during storage and prior to performing a procedure to deploy a tissue anchor.

The needle may have the pre-formed suture knot sutureform anchor disposed about a distal portion thereof while maintained in the elongate rigid tube 1210. For example, the pre-formed suture knot sutureform anchor may be formed of one or more sutures configured in a coiled sutureform (see FIG. 13C) having a plurality of winds/turns around the needle over a portion of the needle that is associated with a longitudinal slot in the needle that runs from the distal end thereof. Although the term “sutureform” is used herein, it should be understood that such components/forms may comprise suture, wire, or any other elongate material wrapped or formed in a desired configuration. The coiled sutureform can be provided or shipped disposed around the needle. In some examples, two tether tails extend from the coiled sutureform. FIG. 12 shows the tether tails 160a, 160b of the tether 150 extending through the lumen of the needle and/or through a passageway of the plunger 1240 and may exit the plunger 1240 at a proximal end portion thereof. The coiled sutureform may advantageously be configured to be formed into a suture-type tissue anchor (referred to herein as a “bulky-knot”) in connection with an anchor-deployment procedure, as described in more detail below. The coiled sutureform can be configurable to a knot/deployed configuration by approximating opposite ends of the coiled portion thereof towards each other to form one or more loops.

The tether delivery system 1200 can further include a suture/tether catch mechanism (not shown) coupled to the plunger 1240 at a proximal end of the tether delivery system 1200, which may be configured to releasably hold or secure the tether 150, such as the tether tails 160a, 16ob, extending through the tether delivery system 1200 during delivery of a tissue anchor as described herein. The suture catch can be used to hold the tether 150 with a friction fit or with a clamping force and can have a lock that can be released after the tissue anchor has been deployed/formed into a bulky-knot, as described herein.

As described herein, the tether delivery system 1200 can be used in beating heart repair procedures. In some examples, the elongate rigid tube 1210 of the tether delivery system 1200 can be configured to extend and contract with the beating of the heart. During systolic contraction, the median axis of the heart generally shortens. For example, the distance from the apex of the heart to the valve leaflets can vary by about 1 centimeter (cm) to about 2 centimeters (cm) with each heartbeat in some patients. In some examples, the length of the elongate rigid tube 1210 that protrudes from the handle 1220 can change with the length of the median axis of the heart. That is, distal end of the elongate rigid tube 1210 can be configured to be floating such that the shaft can extend and retract with the beat of the heart so as to maintain contact with the target mitral valve leaflet.

Implementation of a repair procedure utilizing the tether delivery system 1200 can be performed in conjunction with certain imaging technology designed to provide visibility of the elongate rigid tube 1210 according to a certain imaging modality, such as echo imaging. Advancement of the tether delivery system 1200 may be performed in conjunction with echo imaging, direct visualization (e.g., direct transblood visualization), and/or any other suitable remote visualization technique/modality. With respect to cardiac procedures, for example, the tether delivery system 1200 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). Typical procedures that can be implemented using echo guidance are set forth in Suematsu, Y., J. Thorac. Cardiovasc. Surg. 2005; 130:1348-56 (“Suematsu”), the entire disclosure of which is incorporated herein by reference.

FIGS. 13A through 13E show various steps for delivering the tether 150 using the tether delivery system 1200 described with reference to FIG. 12. FIG. 13A is a cutaway view of a portion of the heart, and the tether delivery system 1200 disposed at least partially within a chamber of a heart in accordance with one or more examples. According to some implementations of repair procedures, an incision into the heart wall of the appropriate ventricle of the heart is made. FIG. 13A shows the tether delivery system 1200 at least partially positioned within the left ventricle 3. For instance, an introducer port device 1300 containing one or more fluid-retention valves to prevent blood loss from and/or air entry into the left ventricle 3, may be inserted into the site of entry. Once the introducer port device 1300 is inside the left ventricle 3, the elongate rigid tube 1210 may be advanced through the lumen 1320 of the introducer port device 1300. In some examples, a sheath may be inserted through the introducer port device 1300, through which one or more other instruments are advanced. For instance, an endoscope may first be advanced into the left ventricle 3 to visualize the ventricle 3, the mitral valve 6, and/or the sub-valvular apparatus. By use of an appropriate endoscope, a careful analysis of the malfunctioning mitral valve 6 may be performed.

One or more incisions may be made proximate to the thoracic cavity to provide a surgical field of access. The total number and length of the incisions to be made depend on the number and types of the instruments to be used as well as the procedure(s) to be performed. The incision(s) may advantageously be made in such a manner as to be minimally invasive. As referred to herein, the term “minimally invasive” means in a manner by which an interior organ or tissue may be accessed with relatively little damage being done to the anatomical structure through which entry is sought. For example, a minimally invasive procedure may involve accessing a body cavity by a small incision/opening of, for example, about 5 centimeters (cm) or less made in the skin of the body. The incision may be vertical, horizontal, or slightly curved. If the incision is located along one or more ribs, it may advantageously follow the outline of the rib. The opening may advantageously extend deep enough to allow access to the thoracic cavity between the ribs or under the sternum and is preferably set close to the rib cage and/or diaphragm, dependent on the entry point chosen.

In one example method, the heart may be accessed through one or more openings made by one or more small incision in a portion of the body proximal to the thoracic cavity, such as between one or more of the ribs of the rib cage of a patient, proximate to the xyphoid appendage, or via the abdomen and diaphragm. Access to the thoracic cavity may be sought to allow the insertion and use of one or more thorascopic instruments, while access to the abdomen may be sought to allow the insertion and use of one or more laparoscopic instruments. Insertion of one or more visualizing instruments may then be followed by transdiaphragmatic access to the heart. Additionally, access to the heart may be gained by direct puncture (e.g., via an appropriately sized needle, for instance an 18-gauge needle) of the heart from the xyphoid region. Accordingly, the one or more incisions should be made in such a manner as to provide an appropriate surgical field and access site to the heart in the least invasive manner possible. Access may also be achieved using percutaneous methods, further reducing the invasiveness of the procedure. See, e.g., “Full-Spectrum Cardiac Surgery Through a Minimal Incision Mini-Sternotomy (Lower Half) Technique,” Doty et al., Annals of Thoracic Surgery 1998; 65(2): 573-7 and “Transxiphoid Approach Without Median Sternotomy for the Repair of Atrial Septal Defects,” Barbero-Marcial et al., Annals of Thoracic Surgery 1998; 65(3): 771-4, the entire disclosures of each, which are incorporated herein by reference for all purposes.

Generally, the elongate rigid tube 1210 of the tether delivery system 1200 may be slowly advanced into the introducer 1300 until the atraumatic tip 1214 is distal to the introducer 1300 and/or cannula tip 1230 thereof and entered the left ventricle 3. For example, the introducer 1300 can comprise a hub 1302 and an elongate shaft 1304 extending distally from the hub 1302. The shaft 1304 can comprise at least a portion configured to be disposed within the left ventricle 3, such as through an incision and/or opening 40 formed in the ventricular heart wall 17 of the left ventricle 3. The elongate tube 1210 of the tether delivery system 1200 can be advanced until the atraumatic tip 1214 is positioned distally of the shaft 1304. In so doing, it may be desirable to advance the elongate rigid tube 1210 within the left ventricle 3 in such a way as to avoid traversing areas populated by papillary muscles and/or associated chordae tendineae to avoid entanglement therewith. In order to facilitate or ensure avoidance of such anatomy, imaging technology may advantageously be implemented to provide at least partial visibility of the elongate rigid tube 1210 within the left ventricle 3, as well as certain anatomical features within the ventricle. In some implementations, hybrid imaging technologies may be used, wherein echo imaging is used in combination with a different imaging modality. Multi-imaging modalities may provide improved visibility of anatomical and/or delivery system components.

FIG. 13B shows a close-up view of the elongate rigid tube 1210 of the tether delivery system 1200 inserted into the left ventricle 3 and approximated to a target portion of the annulus adjacent to the mitral valve leaflet 64, which may be a posterior leaflet of the mitral valve 6, in connection with a repair procedure in accordance with one or more examples of the present disclosure. The elongate rigid tube 1210 can be configured to deliver a tissue anchor (not shown; see, e.g., FIGS. 13C through 13E), such as a bulky-knot, to the portion of the annulus adjacent to the valve leaflet 64. It will be understood that the elongate rigid tube 1210 can also deliver a tissue anchor to the anterior mitral valve leaflet. Although the description of FIGS. 13A through 13E below is presented in the context of a mitral valve, it should be understood that the principles disclosed herein are applicable to other valves or biological tissues, such as a tricuspid valve.

With reference to FIGS. 13A through 13E, the elongate rigid tube 1210 can comprise one or more elongate lumens configured to allow delivery of the anchor 110 to the annulus of the mitral valve 6. The elongate rigid tube 1210 can be configured to facilitate performance of one or more functions, such as grasping, suctioning, irrigating, cutting, suturing, or otherwise engaging a valve leaflet. The distal end, or atraumatic tip, 1214 of the elongate rigid tube 1210 can be configured to contact the portion of the annulus adjacent to the mitral valve leaflet 64 without substantially damaging the annulus. For example, during a repair procedure, a handle (e.g., handle 1220) coupled to the elongate rigid tube 1210 can be manipulated in such a manner so that the annulus is contacted with the functional distal portion of the elongate rigid tube 1210 and a repair effectuated.

Echo imaging guidance, such as transesophageal echocardiogram (TEE) (2D and/or 3D), transthoracic echocardiogram (TTE), and/or intracardiac echo (ICE), may be used to assist in the advancement and desired positioning of the elongate rigid tube 1210 within the left ventricle 3. The atraumatic tip 1214 of the elongate rigid tube 1210 can contact a proximal surface (e.g., underside surface with respect to the illustrated orientation of FIGS. 13B through 13E) of the portion of the annulus adjacent to the mitral valve leaflet 64, without or substantially without damaging the annulus. For example, the atraumatic tip 1214 can have a relatively blunt form or configuration. The atraumatic tip 1214 can be configured to maintain contact with the proximal side of the annulus as the heart beats to facilitate reliable delivery of the anchor 110 to the target site on the annulus.

In some examples, one or more perforation devices 1230 (e.g., needle(s)) can be delivered through a working lumen (not shown) of the elongate rigid tube 1210 to the portion of the mitral valve annulus adjacent to the valve leaflet 64 to puncture the portion of the annulus and project a sutureform 190 including a plurality of winds of suture about a distal portion of a needle 1230 into the left atrium 2 (see FIG. 13C), wherein the sutureform 190 is deployed to form the bulky-knot anchor 110 shown in FIGS. 13D and 13E. As described herein, in some instances, the first anchor 110 and the tether 150 can be an integral piece formed using a suture 102. For example a portion of the suture 102 can form the sutureform 190. Another portion of the suture 102 can form the tether 150, including the tether tails 160a, 16ob of the tether 150. For example, as shown in FIG. 13C, the needle 1230 can comprise a slot and can be deployed from the distal end of the elongate rigid tube 1210, thereby puncturing the annulus and projecting into the left atrium 2, wherein the slotted needle 1230 is wrapped with a sutureform 190 in a particular configuration (see PCT Application No. PCT/US2012/043761, published as WO 2013/003228 A1, for further detail regarding example suture wrapping configurations and needles for use in suture anchor deployment devices and methods, the entirety of which is incorporated herein by reference). In some instances, a pusher or hollow guide wire (not shown) is provided on or at least partially around the needle 1230 within the elongate rigid tube 1210, such that the needle may be withdrawn, leaving the pusher and wound sutureform 190. When a withdrawal force is applied to the sutureform 190 using the pusher, the sutureform 190 may form a bulky-knot-type anchor (e.g., the anchor 110), after which the pusher may be withdrawn, leaving the bulky-knot-type anchor to anchor the tether tails 160a, 160b to the annulus.

FIG. 13B shows the elongate rigid tube 1210 of the tether delivery system 1200 positioned on the mitral valve annulus adjacent to the mitral valve leaflet 64. For example, the target site of the annulus may be slowly approached from the ventricle side thereof by advancing the distal end of elongate rigid tube 1210 along or near to the posterior wall of the left ventricle 3 without contacting the ventricle wall. Successful targeting and contacting of the target location on the annulus can depend at least in part on accurate visualization of the elongate rigid tube 1210 and/or atraumatic tip 1214 throughout the process of advancing the atraumatic tip 1214 to the target site. Generally, echocardiographic equipment may be used to provide the necessary or desired intra-operative visualization of the elongate rigid tube 1210 and/or atraumatic tip 1214.

Once the atraumatic tip 1214 is positioned in the desired position, the distal end of the elongate rigid tube 1210 and the atraumatic tip 1214 may be used to drape, or “tent,” the annulus to better secure the atraumatic tip 1214 in the desired position, as shown in FIG. 13B. Draping/tenting may advantageously facilitate contact of the atraumatic tip 1214 with the annulus throughout one or more cardiac cycles, to thereby provide more secure or proper deployment of leaflet anchor(s). Navigation of the atraumatic tip 1214 to the desired location on the underside of the mitral valve annulus may be assisted using echo imaging, which may be relied upon to confirm correct positioning of the atraumatic tip 1214 prior to anchor/knot deployment.

With the elongate rigid tube 1210 positioned against the mitral valve annulus adjacent to the mitral valve leaflet 64, the plunger 1240 of the tether delivery system 1200 can be actuated to move the needle 1230 and a pusher disposed within the elongate rigid tube 1210, such that the coiled sutureform portion 190 of the suture anchor slides off the needle 1230, as shown in FIG. 13C. As the plunger 1240 is actuated, a distal piercing portion of the needle 1230 punctures the annulus and forms an opening in the annulus. FIG. 13C shows a close-up view of the distal portion of the elongate rigid tube 1210 showing the needle 1230 and tissue anchor sutureform 190 projected therefrom through the annulus in accordance with one or more examples. In some examples, the needle 1230 is projected a distance of between about 0.2 to about 0.3 inches (about 5 to about 8 millimeters (mm)), or less, distally beyond the distal end of the elongate rigid tube 1210 (e.g., beyond the atraumatic tip 1214). In some examples, the needle 1230 is projected a distance of between about 0.15 to about 0.4 inches (about 4 to about 10 millimeters (mm)). In some examples, the needle 1230 is projected a distance of about 1 inch (about 25 millimeters (mm)), or greater. In some examples, the needle 1230 extends until the distal tip of the needle and the entire coiled sutureform 190 extend through the annulus. While the needle 1230 and sutureform 190 are projected into the side of the annulus oriented toward the left atrium 2, the elongate rigid tube 1210 and atraumatic tip 1214 advantageously remain entirely on the side of the annulus oriented toward the left ventricle 3.

As the pusher (not shown) within the tissue anchor delivery device elongate rigid tube 1210 is moved distally, a distal end of the pusher advantageously moves or pushes the distal coiled sutureform 190 (e.g., pre-deployment coiled portion of the suture anchor) over the distal end of the needle 1230 and further within the left atrium 2 of the heart on a distal side of the mitral valve annulus adjacent to the mitral valve leaflet 64, such that the sutureform 190 extends distally beyond a distal end of the needle 1230. For example, in some examples, at least half a length of the sutureform 190 extends beyond the distal end of the needle 1230. In some examples, at least three quarters of the length of the sutureform 190 extends beyond the distal end of the needle 1230. In some examples, the entire coiled sutureform 190 extends beyond the distal end of the needle 1230.

After the sutureform 190 has been pushed off the needle 1230, pulling one or more of the tether tail(s) 160a, 160b (e.g., strands extending from the coiled portion of the tether) associated with the tissue anchor 110 proximally can cause the sutureform 190 to form the anchor 110 (e.g., a bulky-knot anchor), as shown in FIG. 13D, which provides a close-up view of the formed anchor 110 on the side of the annulus oriented toward the left atrium 2. For example, the bulky-knot suture anchor may be formed by approximating opposite ends of the coils of the sutureform 190 (see FIG. 13C) towards each other to form one or more loops. After the sutureform 190 has been formed into the bulky-knot, the tether delivery system 1200 can be withdrawn proximally, leaving the anchor 110 disposed on the distal atrial side of the annulus.

FIG. 13E shows a cutaway view of the heart with the deployed anchor 110 and the elongate rigid tube 1210 being withdrawn from the mitral valve annulus after the anchor 110 has been formed. The tether tails 160a, 160b extend proximally from the anchor 110. In some examples, the two tether tails 160a, 16ob may extend from the proximal side of the annulus, such as the side of the annulus oriented toward the left ventricle 3. The tether tails 160a, 16ob can extend out of the heart 1 through the opening 40 formed in the ventricular wall. For example, the elongate rigid tube 1210 can be slid/withdrawn over the tether tails 160a, 16ob. The tether tails 160a, 16ob can advantageously be tensioned to facilitate desired reshaping of the left ventricle. The tether tails 160a, 160b coupled to the anchor 110 may be secured at the desired tension using a surgical pad as described herein. One or more knots (e.g., a knot stack) or other suture fixation mechanism(s) or device(s) may be implemented to hold the tether tails 160a, 16ob at the desired tension and to the surgical pad. With the tether tails 160a, 16ob fixed to the ventricle wall, a ventricular portion of the tether tails 160a, 16ob may advantageously function to tether the mitral valve annulus in a desired manner and at a desired tension, facilitating reshaping of left ventricular wall.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.
Example 1: A tether system comprising a first anchor configured to be coupled to an annulus of a heart valve that is between a heart atrium and a heart ventricle, a second anchor configured to be positioned over an externally oriented surface of a portion of a ventricular heart wall of the heart ventricle between an apex and a basal portion of the ventricular heart wall, and a tether configured to couple the first anchor and the second anchor to one another.
Example 2: The system of any of the examples described herein, in particular example 1, wherein the portion of the ventricular heart wall is in a mid-portion of the ventricular heart wall.
Example 3: The system of any of the examples described herein, in particular example 1, wherein the portion of the ventricular heart wall is between an apical portion and a mid-portion of the ventricular heart wall.
Example 4: The system of any of the examples described herein, in particular example 1, wherein the portion of the ventricular heart wall is adjacent to a portion of the ventricular heart wall from which a papillary muscle extends.
Example 5: The system of any of the examples described herein, in particular example 4, wherein the portion of the ventricular heart wall is adjacent to a portion of the ventricular heart wall from which an anterolateral papillary muscle extends.
Example 6: The system of any of the examples described herein, in particular examples 1 to 5, wherein the first anchor is configured to be coupled to an annulus of a mitral valve and the heart ventricle is a left ventricle.
Example 7: The system of any of the examples described herein, in particular example 6, wherein the first anchor is configured to be coupled to a portion of the annulus adjacent to a posterior leaflet of the mitral valve.
Example 8: The system of any of the examples described herein, in particular example 6, wherein the first anchor is configured to be coupled to a portion of the annulus adjacent to an anterior leaflet of the mitral valve.
Example 9: The system of any of the examples described herein, in particular examples 1 to 8, further comprising an elongate tube configured to receive at least a portion of the tether extending within the heart ventricle.
Example 10: The system of any of the examples described herein, in particular examples 1 to 9, wherein the tether comprises a suture.
Example 11: The system of any of the examples described herein, in particular example 10, wherein the first anchor comprises at least a portion formed using the suture.
Example 12: The system of any of the examples described herein, in particular example 11, wherein the first anchor comprises a bulky-knot.
Example 13: The system of any one of any of the examples described herein, in particular examples 1 to 12, wherein the tether is configured to extend through the ventricular heart wall to couple to the second anchor.
Example 14: A tether system comprising a first anchor configured to be coupled to a septal wall of a heart ventricle, a second anchor configured to be positioned over an externally oriented surface of a portion of a ventricular heart wall of the heart ventricle, and a tether configured to couple the first anchor and the second anchor to one another.
Example 15: The system of any of the examples described herein, in particular example 14, wherein the first anchor is configured to be within the septal wall.
Example 16: The system of any of the examples described herein, in particular example 14, wherein the first anchor is configured to be over a surface of the septal wall having an orientation opposite relative to that oriented toward the heart ventricle.
Example 17: The system of any of the examples described herein, in particular examples 14 to 16, wherein the first anchor is configured to be coupled to a basal portion of the septal wall.
Example 18: The system of any of the examples described herein, in particular examples 14 to 16, wherein the first anchor is configured to be coupled to a mid-portion of the septal wall.
Example 19: The system of any of the examples described herein, in particular examples 14 to 18, wherein the second anchor is configured to be coupled to a mid-portion of the ventricular heart wall.
Example 20: The system of any of the examples described herein, in particular examples 14 to 19, wherein the tether is configured to extend directly between the first anchor and the second anchor.
Example 21: The system of any of the examples described herein, in particular examples 14 to 19, wherein the tether comprises a portion configured to form a loop around respective portions of papillary muscles and chordae tendineae in the heart ventricle.
Example 22: The system of any of the examples described herein, in particular example 21, wherein the heart ventricle is a left ventricle, and wherein the portion of the tether comprises is configured to form a loop around both a posteromedial papillary muscle or chordae tendineae coupled to the posteromedial papillary muscle, and an anterolateral papillary muscle or chordae tendineae coupled to the posteromedial papillary muscle.
Example 23: The system of any of the examples described herein, in particular examples 14 to 22, further comprising an elongate tube configured to receive at least a portion of the tether extending within the heart ventricle.
Example 24: The system of any of the examples described herein, in particular examples 14 to 23, wherein the tether comprises a suture.
Example 25: The system of any of the examples described herein, in particular example 24, wherein the first anchor comprises at least a portion formed using the suture.
Example 26: The system of any of the examples described herein, in particular example 25, wherein the first anchor comprises a bulky-knot.
Example 27: The system of any of the examples described herein, in particular examples 14 to 27, wherein the tether is configured to extend through the ventricular heart wall to couple to the second anchor.
Example 28: A tether system comprising a first anchor configured to be coupled to a papillary muscle of a heart ventricle, a second anchor configured to be positioned over an externally oriented surface of an apical portion of a ventricular heart wall of the heart ventricle, and a tether configured to couple the first anchor and the second anchor to one another.
Example 29: The system of any of the examples described herein, in particular example 28, wherein the heart ventricle is a left ventricle and the first anchor is configured to be coupled to an anterolateral papillary muscle.
Example 30: The system of any of the examples described herein, in particular example 28 or 29, wherein the second anchor is configured to be coupled to an externally oriented surface of an apex of the ventricular heart wall.
Example 31: The system of any of the examples described herein, in particular examples 28 to 30, further comprising an elongate tube configured to receive at least a portion of the tether extending within the heart ventricle.
Example 32: The system of any of the examples described herein, in particular examples 28 to 31, wherein the tether comprises a suture.
Example 33: The system of any of the examples described herein, in particular example 32, wherein the first anchor comprises at least a portion formed using the suture.
Example 34: The system of any of the examples described herein, in particular example 33, wherein the first anchor comprises a bulky-knot.
Example 35: The system of any of the examples described herein, in particular examples 28 to 34, wherein the tether is configured to extend through the ventricular heart wall to couple to the second anchor.
Example 36: A system comprising a first anchor configured to be positioned over an externally oriented surface of a first portion of a ventricular heart wall of a heart ventricle, a second anchor configured to be positioned over an externally oriented surface of a second portion of the ventricular heart wall of the heart ventricle, and a tether configured to couple the first anchor and the second anchor to one another, the tether comprising a portion configured to form at least a partial loop around a papillary muscle of the heart ventricle or chordae tendineae coupled to the papillary muscle.
Example 37: The system of any of the examples described herein, in particular example 36, wherein the first portion of the ventricular heart wall is above the second portion of the ventricular heart wall.
Example 38: The system of any of the examples described herein, in particular example 36 or 37, wherein at least one of the first portion and the second portion are in a mid-portion of the ventricular heart wall.
Example 39: The system of any of the examples described herein, in particular example 36 or 37, wherein at least one of the first portion and the second portion are in an apical portion of the ventricular heart wall.
Example 40: The system of any one of any of the examples described herein, in particular examples 36 to 39, wherein the heart ventricle is a left ventricle and the portion of tether is configured to form at least a partial loop around a posteromedial papillary muscle or chordae tendineae coupled to the posteromedial papillary muscle.
Example 41: The system of any of the examples described herein, in particular example 40, wherein the portion of the tether forms a partial loop around the posteromedial papillary muscle or chordae tendineae coupled to the posteromedial papillary muscle.
Example 42: The system of any of the examples described herein, in particular examples 36 to 41, further comprising an elongate tube configured to receive at least a portion of the tether extending within the heart ventricle.
Example 43: The system of any of the examples described herein, in particular examples 36 to 42, wherein the tether comprises a suture.
Example 44: The system of any of the examples described herein, in particular example 43, wherein the first anchor comprises at least a portion formed using the suture.
Example 45: The system of any of the examples described herein, in particular example 44, wherein the first anchor comprises a bulky-knot.
Example 46: The system of any of the examples described herein, in particular examples 36 to 45, wherein the tether is configured to extend through the ventricular heart wall to couple to the second anchor.
Example 47: A method comprising coupling a first anchor to an annulus of a heart valve that is between a heart atrium and a heart ventricle, extending a portion of a tether within the heart ventricle from the first anchor to a portion of a ventricular heart wall of the heart ventricle between an apex and a basal portion of the ventricular wall, positioning a second anchor over an externally oriented surface of the portion of the ventricular heart wall, and coupling the tether to the second anchor while the second anchor is over the externally oriented surface of the ventricular heart wall.
Example 48: The method of any of the examples described herein, in particular example 47, wherein extending the portion of the tether from the first anchor to the portion of the heart ventricular heart comprises extending the tether from the first anchor to a mid-portion of the ventricular heart wall.
Example 49: The method of any of the examples described herein, in particular example 47, wherein extending the portion of the tether from the first anchor to the portion of the ventricular heart wall comprises extending the tether from the first anchor to a portion of the ventricular heart wall between an apical portion and a mid-portion of the ventricular heart wall.
Example 50: The method of any of the examples described herein, in particular example 47, wherein extending the portion of the tether from the first anchor to the portion of the ventricular heart wall comprises extending the tether from the first anchor to a portion of the ventricular heart wall adjacent to a papillary muscle.
Example 51: The method of any of the examples described herein, in particular example 50, wherein extending the portion of the tether from the first anchor to the portion of the ventricular heart wall comprises extending the tether from the first anchor to a portion of the ventricular heart wall adjacent to anterolateral papillary muscle.
Example 52: The method of any of the examples described herein, in particular examples 47 to 51, wherein coupling the first anchor to the annulus of the heart valve comprises coupling the first anchor to a mitral valve annulus.
Example 53: The method of any of the examples described herein, in particular example 52, wherein coupling the first anchor to the annulus of the heart valve comprises coupling the first anchor to a portion of the mitral valve annulus adjacent to a posterior leaflet.
Example 54: The method of any of the examples described herein, in particular example 52, wherein coupling the first anchor to the annulus of the heart valve comprises coupling the first anchor to a portion of the mitral valve annulus adjacent to an anterior leaflet.
Example 55: The method of any of the examples described herein, in particular examples 47 to 54, further comprising advancing an elongate tube along at least a portion of the tether within the heart ventricle.

The above method(s) can be performed on a living animal or on a simulation, such as on a cadaver, cadaver heart, anthropomorphic ghost, simulator (e.g., with body parts, heart, tissue, etc. being simulated).
Example 56: A method comprising coupling a first anchor to a septal wall of a heart ventricle, extending a portion of a tether within the heart ventricle from the first anchor to a portion of a ventricular heart wall of the heart ventricle, positioning a second anchor over an externally oriented surface of the portion of the ventricular heart wall, and coupling the tether to the second anchor while the second anchor is over the externally oriented surface of the ventricular heart wall.
Example 57: The method of any of the examples described herein, in particular example 56, wherein coupling the first anchor to the septal wall comprises embedding the first anchor within the septal wall.
Example 58: The method of any of the examples described herein, in particular example 56, wherein coupling the first anchor to the septal wall comprises positioning the first anchor over a surface of the septal wall having an opposing orientation relative to that oriented toward the heart ventricle.
Example 59: The method of any of the examples described herein, in particular examples 56 to 58, wherein coupling the first anchor to the septal wall comprises coupling the first anchor to a basal portion of the septal wall.
Example 60: The method of any of the examples described herein, in particular examples 56 to 58, wherein coupling the first anchor to the septal wall comprises coupling the first anchor to a mid-portion of the septal wall.
Example 61: The method of any of the examples described herein, in particular examples 56 to 60, wherein extending the portion of the tether from the first anchor to the portion of the ventricular heart wall of the heart ventricle comprises extending the portion of the tether from the first anchor to a mid-portion of the ventricular heart wall.
Example 62: The method of any of the examples described herein, in particular examples 56 to 61, wherein extending the portion of the tether from the first anchor to the portion of the ventricular heart wall of the heart ventricle comprises extending the portion of the tether directly from the first anchor to the second anchor.
Example 63: The method of any of the examples described herein, in particular examples 56 to 61, wherein extending the portion of the tether from the first anchor to the portion of the ventricular heart wall of the heart ventricle comprises looping the tether around both a posteromedial papillary muscle or chordae tendineae coupled to the posteromedial papillary muscle, and an anterolateral papillary muscle or chordae tendineae coupled to the posteromedial papillary muscle.
Example 64: The method of any of the examples described herein, in particular examples 56 to 63, further comprising advancing an elongate tube along at least a portion of the tether within the heart ventricle.

The above method(s) can be performed on a living animal or on a simulation, such as on a cadaver, cadaver heart, anthropomorphic ghost, simulator (e.g., with body parts, heart, tissue, etc. being simulated).
Example 65: A method comprising coupling a first anchor to a papillary muscle of a heart ventricle, extending a portion of a tether within the heart ventricle from the first anchor to an apical portion of a ventricular heart wall of the heart ventricle, positioning a second anchor over an externally oriented surface of the apical portion of the ventricular heart wall, and coupling the tether to the second anchor while the second anchor is over the externally oriented surface of the apical portion of the ventricular heart wall.
Example 66: The method of any of the examples described herein, in particular example 65, wherein coupling the first anchor to the papillary muscle comprises coupling the first anchor to an anterolateral papillary muscle.
Example 67: The method of any of the examples described herein, in particular example 65 or 66, wherein extending the portion of the tether within the heart ventricle from the first anchor to the apical portion of the ventricular heart wall comprises extending the portion of the tether within the heart ventricle from the first anchor to an apex of the ventricular heart wall.
Example 68: The method of any of the examples described herein, in particular examples 65 to 67, further comprising advancing an elongate tube along at least a portion of the tether within the heart ventricle.

The above method(s) can be performed on a living animal or on a simulation, such as on a cadaver, cadaver heart, anthropomorphic ghost, simulator (e.g., with body parts, heart, tissue, etc. being simulated).
Example 69: A method comprising coupling a first anchor to a first portion of a ventricular heart wall of a heart ventricle, and extending a portion of a tether within the heart ventricle from the first anchor to a second portion of the ventricular heart wall of the heart ventricle and forming at least a partial loop, using the tether, around a papillary muscle of the heart ventricle or chordae tendineae coupled to the papillary muscle. The method can include positioning a second anchor over an externally oriented surface of the second portion of the ventricular heart wall, and coupling the tether to the second anchor while the second anchor is over the externally oriented surface of the ventricular heart wall.
Example 70: The method of any of the examples described herein, in particular example 69, wherein extending the portion of the tether within the heart ventricle from the first anchor to the second portion of the ventricular heart wall comprises extending the portion of the tether within the heart ventricle from the first anchor to a second portion of the ventricular heart wall that is lower on the ventricular heart wall than the first portion.
Example 71: The method of any of the examples described herein, in particular example 69 or 70, wherein coupling the first anchor to the first portion of the ventricular heart wall comprises coupling the first anchor to a first mid-portion of the ventricular heart wall.
Example 72: The method of any of the examples described herein, in particular example 71, wherein extending the portion of the tether within the heart ventricle from the first anchor to the second portion of the ventricular heart wall comprises extending the portion of the tether within the heart ventricle from the first anchor to a second mid-portion of the ventricular heart wall.
Example 73: The method of any of the examples described herein, in particular example 71, wherein extending the portion of the tether within the heart ventricle from the first anchor to the second portion of the ventricular heart wall comprises extending the portion of the tether within the heart ventricle from the first anchor to a second apical portion of the ventricular heart wall.
Example 74: The method of any of the examples described herein, in particular example 69, wherein:
coupling the first anchor to the first portion of the ventricular heart wall comprises coupling the first anchor to a first apical portion of the ventricular heart wall; and
extending the portion of the tether within the heart ventricle from the first anchor to the second portion of the ventricular heart wall comprises extending the portion of the tether within the heart ventricle from the first anchor to a second apical portion of the ventricular heart wall.
Example 75: The method of any of the examples described herein, in particular examples 69 to 75, wherein forming at least a partial loop, using the tether, around the papillary muscle comprises forming at least a partial loop, using the tether, around a posteromedial papillary muscle or chordae tendineae coupled to the posteromedial papillary muscle.
Example 76: The method of any of the examples described herein, in particular examples 69 to 75, wherein forming at least a partial loop, using the tether, around the posteromedial papillary muscle comprises forming a partial loop around the posteromedial papillary muscle or chordae tendineae coupled to the posteromedial papillary muscle.
Example 77: The method of any of the examples described herein, in particular examples 69 to 76, further comprising advancing an elongate tube along at least a portion of the tether within the heart ventricle.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method comprising:
coupling a first anchor to an annulus of a heart valve that is between a heart atrium and a heart ventricle;
extending a portion of a tether within the heart ventricle from the first anchor to a portion of a ventricular heart wall of the heart ventricle between an apex and a basal portion of the ventricular heart wall;
positioning a second anchor over an externally oriented surface of the portion of the ventricular heart wall; and
coupling the tether to the second anchor while the second anchor is over the externally oriented surface of the ventricular heart wall.

2. The method of claim 1, wherein extending the portion of the tether from the first anchor to the portion of the ventricular heart wall comprises extending the tether from the first anchor to a mid-portion of the ventricular heart wall.

3. The method of claim 1, wherein extending the portion of the tether from the first anchor to the portion of the ventricular heart wall comprises extending the tether from the first anchor to a portion of the ventricular heart wall between an apical portion and a mid-portion of the ventricular heart wall.

4. The method of claim 1, wherein extending the portion of the tether from the first anchor to the portion of the ventricular heart wall comprises extending the tether from the first anchor to a portion of the ventricular heart wall adjacent to a papillary muscle.

5. The method of claim 4, wherein extending the portion of the tether from the first anchor to the portion of the ventricular heart wall comprises extending the tether from the first anchor to a portion of the ventricular heart wall adjacent to anterolateral papillary muscle.

6. The method of claim 1, wherein coupling the first anchor to the annulus of the heart valve comprises coupling the first anchor to a mitral valve annulus.

7. The method of claim 6, wherein coupling the first anchor to the annulus of the heart valve comprises coupling the first anchor to a portion of the mitral valve annulus adjacent to a posterior leaflet.

8. The method of claim 6, wherein coupling the first anchor to the annulus of the heart valve comprises coupling the first anchor to a portion of the mitral valve annulus adjacent to an anterior leaflet.

9. A method comprising:
anchoring a first portion of a tether to a portion of an annulus of a heart valve that is between a heart atrium and a heart ventricle;
extending a medial portion of the tether from the portion of the annulus through the heart ventricle to a portion of a ventricular heart wall of the heart ventricle between an apex and a basal portion of the ventricular heart wall; and
anchoring a second portion of the tether to the portion of the ventricular heart wall to pull the portion of the ventricular heart wall toward the portion of the annulus and provide reversal in remodeling of the ventricular heart wall.

10. The method of claim 9, wherein extending the medial portion of the tether comprises extending the medial portion of the tether from the portion of the annulus through the heart ventricle to a mid-portion of the ventricular heart wall.

11. The method of claim 9, wherein extending the medial portion of the tether comprises extending the medial portion of the tether from the portion of the annulus through the heart ventricle to a portion of the ventricular heart wall between an apical portion and a mid-portion of the ventricular heart wall.

12. The method of claim 9, wherein extending the medial portion of the tether comprises extending the medial portion of the tether from the portion of the annulus through the heart ventricle to a portion of the ventricular heart wall adjacent to a papillary muscle to avoid traversing areas populated by papillary muscles.

13. The method of claim 9 further comprising advancing an elongate tube along at least a portion of the medial portion of the tether to prevent contact between the at least a portion of the medial portion of the tether and heart tissue.

14. A method comprising:
providing an anchor over an atrium-facing surface of an annulus of a heart valve;
tethering the anchor through a heart ventricle to a portion of a ventricular heart wall of the heart ventricle between an apex and a basal portion of the ventricular heart wall; and
anchoring the tether to the portion of the ventricular heart wall.

15. The method of claim 14, wherein providing the anchor over the atrium-facing surface of the annulus comprises forming the anchor using a portion of a suture.

16. The method of claim 15, wherein forming the anchor using the portion of the suture comprises forming a bulky-knot using the portion of the suture.

17. The method of claim 15, wherein tethering the anchor through the heart ventricle to the portion of the ventricular heart wall comprises extending another portion of the suture through the annulus and the heart ventricle to the portion of the ventricular heart wall.

18. The method of claim 14, wherein tethering the anchor through the heart ventricle to the portion of the ventricular heart wall comprises tethering the anchor through the heart ventricle to a mid-portion of the ventricular heart wall.

19. The method of claim 14, wherein tethering the anchor through the heart ventricle to the portion of the ventricular heart wall comprises tethering the anchor through the heart ventricle to a portion of the ventricular heart wall between an apical portion and a mid-portion of the ventricular heart wall.

20. The method of claim 14, wherein tethering the anchor through the heart ventricle to the portion of the ventricular heart wall comprises tethering the anchor through the heart ventricle to a portion of the ventricular heart wall adjacent to a papillary muscle.
